(19) United States
(12) Patent Application Publication
(10) Pub. No.: US 2003/0181436 A1
(43) Pub. Date: Sep. 25, 2003

(54) Treatment for AIDS, HIV, and other related diseases and compounds for use therewith

(76) Inventor: George P. Sakalosky, Gatlinburg, TN (US)

Correspondence Address: DORSEY & WHITNEY LLP, INTELLECTUAL PROPERTY DEPARTMENT, 50 SOUTH SIXTH STREET, MINNEAPOLIS, MN, 55402-1498, US

(21) Appl. No.: 10/293,054

(22) Filed: Nov. 13, 2002

Related U.S. Application Data
(60) Provisional application No. 60/338,225, filed on Nov. 13, 2001.

(51) Int. Cl. A61K 31/555

(52) U.S. Cl. 514/184

(57) ABSTRACT

A treatment for HIV, AIDS, and AIDS related diseases and compounds for use therewith are described. The compounds treat HIV, AIDS, and AIDS related diseases by inhibiting or preventing the virus from undergoing reverse transcription, post reverse transcription, integration, and/or post integration. The compounds also display activity against latently infected cells. In particular, the compounds each contain strontium; iodine; ascorbic acid; and a diamagnetic ion, either zinc, bismuth, or potassium.

### PRIORITY CLAIM

This application claims priority to U.S. Provisional Application No. 60/338,225, filed on Nov. 13, 2001, entitled “Treatment for AIDS, HIV and Other Related Diseases an Compounds for Use Therewith”, the contents of which are incorporated herein by reference in its entirety.

### FIELD

The present invention relates to treatment of human immunodeficiency virus (HIV), acquired immunodeficiency syndrome (AIDS), and AIDS related diseases. More particularly, it relates to a method for preventing, treating and curing HIV, AIDS, AIDS related diseases, and to compounds for use in the treatment. The present invention provides a new and unique approach to inhibiting or preventing HIV virus replication, as well as providing compounds which have therapeutic activity against latently infected cells.

### BACKGROUND

Human immunodeficiency virus (HIV) has been etiologically linked to acquired immunodeficiency syndrome (AIDS). AIDS is a retroviral disease resulting in an ineffective immune response. Individuals infected with HIV are initially asymptomatic, however such persons typically develop AIDS related complex (ARC) followed by AIDS. The resulting ineffective immune response allows opportunistic infections to invade an individual which may be eventually fatal.

The HIV lifecycle begins with a virion attaching itself to a host T-4 lymphocyte immune cell by binding with a CD4 receptor on the host cell. The virion then sheds it glycoprotein coat and penetrates into the membrane of the host cell. Ribonucleic acid (RNA) is then released and undergoes reverse transcription into DNA. The virion enzyme in HIV, reverse transcriptase, catalyzes the conversion of viral RNA into single-stranded DNA. The viral RNA is degraded and a second strand of DNA is created. The viral DNA then enters the host cell nucleus where the enzyme integrase catalyzes its integration into the genetic material of the cell. HIV may persist in a latent state for many years once the viral DNA has been integrated into the genetic material of the host cell. Infected individuals must presently remain on antiviral therapy for life as a result of latently infected cells. The ability of HIV to persist in certain latently infected cells has also been a major obstacle to finding a cure for HIV.

Activation of the host cells results in the transcription of viral DNA into messenger RNA (mRNA), which is then translated into the precursor of a gag pol fusion polyprotein. The HIV protease enzyme then cleaves the polyprotein, yielding mature viral proteins. HIV protease is responsible for regulating numerous cleavage events allowing the virus particles to mature into a virus that is capable of full infectivity.

The virus continues to reproduce while the immune response system continues to weaken and unless the HIV lifecycle is interrupted by treatment, the virus infection spreads throughout the body. This eventually results in the destruction of the body's immune system and allows opportunistic infections to set in.

There are currently three types of antiviral medications used to inhibit HIV: nucleoside analog reverse transcriptase inhibitors, non-nucleoside reverse transcriptase inhibitors, and protease inhibitors. Nucleoside analog reverse transcriptase inhibitors incorporate into the viral DNA thereby blocking further elongation of the DNA and halting viral replication. Non-nucleoside reverse transcriptase inhibitors block viral DNA replication by interfering with the action of the reverse transcriptase. Protease inhibitors inhibit the HIV protease in infected cells.

Currently these drugs are administered simultaneously, rather than sequentially, in three-drug combinations called cocktails. While drug cocktails can suppress viral replication, they do not cure HIV or AIDS. Additionally, they are not effective in all patients and the virus can develop resistance to the drugs used in the cocktails. Each of the current drugs possess potential side effects including headache, nausea, vomiting, dizziness, anemia, fatigue, diarrhea, rash, hyperglycemia, kidney stones, changes in liver enzyme levels, and development of diabetes or the exacerbation of existing cases of diabetes.

Therefore, a need exists to overcome one of more of the above-identified disadvantages of the current treatments and therapeutics.

### SUMMARY

The present invention relates to a method for treating AIDS, HIV, and AIDS related diseases and packaged pharmaceuticals including the compounds of the present invention for use in those treatments. The compounds of the present invention have unexpectedly been found to be inhibitors of HIV virus replication in human blood cells, and have therapeutic activity against latently infected cells. Unlike, current AIDS therapies which have not been able to eliminate the reservoirs of latently infected cells, the present invention advantageously provides this ability. Latently infected cells are responsible for virus re-bound and disease progression after a patient discontinues treatment or the virus becomes resistant to the treatment being used. It is these latently infected cells which have been a major obstacle to discovering a cure for HIV. The present invention, therefore, provides therapeutic compounds and methods which overcome this obstacle.

Compounds of the invention have unique antiviral targets as shown by studies performed with such therapeutic compounds. In one embodiment of the present invention, the antiviral target for the HIV and AIDS therapeutics is either reverse transcriptase or protease. The present invention is also directed to integrase or post-integration, and post-reverse transcription, as well as reverse transcriptase, as antiviral targets as is shown in the following detailed description of the invention and experimental sections.

In one aspect, the present invention relates to methods for treating human immunodeficiency virus (HIV), acquired immunodeficiency syndrome (AIDS), AIDS related complex (ARC), or AIDS related diseases. The methods include the administration of a therapeutically effective amount of an iodiscorbate and pharmaceutically acceptable salts thereof to a subject in need thereof. In certain embodiments, the iodiscorbate has a general formula of XISrC₆H₅O₆, wherein X can be either bismuth, potassium, or zinc. Consequently, iodiscorbates of the invention include, for example, BiISrC₆H₅O₆, K₂₁SrC₆H₅O₆, and ZnISrC₆H₅O₆. Additionally, the iodiscorbate is considered as a compound that includes operably bonded iodine, ascorbic acid, strontium, and either bismuth, potassium, or zinc.

The treatments provided by the present invention include the inhibition and/or the prevention viral reverse transcription, viral post-reverse transcription, viral integration, and/or viral post-integration. In one aspect, the subject that utilizes the advantageous administration of the therapeutics iodiscorbate compounds of the invention is a human. The method of the invention can further include administration of protease inhibitors, nucleoside analog reverse transcriptase inhibitors and non-nucleoside reverse transcriptase inhibitors.

In another aspect, the invention pertains to packaged pharmaceutical compositions useful for treating human immunodeficiency virus (HIV), acquired immunodeficiency syndrome (AIDS), AIDS related complex (ARC), or AIDS related diseases in a subject. The packaged pharmaceuticals include a container holding a therapeutically effective amount of an iodiscorbate and pharmaceutically acceptable salts thereof, as described herein and appropriate instructions for using the iodiscorbate for treating the particular condition in the subject. Additionally, the packaged pharmaceuticals can further include protease inhibitors, nucleoside analog reverse transcriptase inhibitors and non-nucleoside reverse transcriptase inhibitors.

### BRIEF DESCRIPTION OF THE FIGURES

FIG. 1 depicts a chemical compound, Bismuth Iodiscorbate, hereinafter referred to as Formula A.

FIG. 2 depicts a second chemical compound, Bismuth Iodiscorbate, hereinafter referred to as Formula B.

FIG. 3 depicts a third chemical compound, Zinc Iodiscorbate, herein referred to as compound Z.

FIG. 4 depicts a fourth chemical compound, Potassium Iodiscorbate, herein referred to as compound K.

FIG. 5 depicts toxicity studies of compound K.

FIG. 6 depicts toxicity studies of compound Z.

FIG. 7 shows the effect of compound Z on TNFα induced ACH-2 cells.

FIG. 8 shows the effect of compound Z on U1/TNFα.

FIG. 9 presents the effects of compound K on TNFα induced ACH-2 cells.

FIG. 10 is another depiction of compound Z on TNFα induced ACH-2 cells.

FIG. 11 presents the effect of compound K on TNFα induced U1 cells.

FIG. 12 presents the effect of compound Z on TNFα induced U1 cells.

FIG. 13 shows the toxicity of compound Z on HeLa.

FIG. 14 shows the toxicity of DMSO (control) on HeLa.

FIG. 15 is a comparison of compound Z cytotoxicity and antiviral efficacy on HeLa cells.

FIG. 16 is a study of compound K.

FIG. 17 is a study of compound Z.

FIG. 18 is a time addition study assay of compound K.

FIG. 19 is a time addition study assay of compound Z.

FIG. 20 depicts compound Z versus CEMSK1.

FIG. 21 shows inhibition of attachment by Chicago Sky Blue.

FIG. 22 shows inhibition of attachment by compound Z.

### DETAILED DESCRIPTION

The features and other details of the invention will now be more particularly described and pointed out in the claims. It will be understood that the particular embodiments of the invention are shown by way of illustration and not as limitations of the invention. The principle features of this invention can be employed in various embodiments without departing from the scope of the invention.

In one aspect, the present invention relates to a method for treating human immunodeficiency virus (HIV). The method includes the administration of a therapeutically effective amount of an iodiscorbate and pharmaceutically acceptable salts thereof to a subject in need thereof.

In another aspect, the present invention relates to a method for treating acquired immunodeficiency syndrome (AIDS). The method includes the administration of a therapeutically effective amount of an iodiscorbate and pharmaceutically acceptable salts thereof to a subject in need thereof.

In still another aspect, the present invention relates to a method for treating AIDS related complex (ARC). The method includes the administration of a therapeutically effective amount of an iodiscorbate and pharmaceutically acceptable salts thereof to a subject in need thereof.

In still yet another aspect, the present invention relates to a method for treating AIDS related diseases. The method includes the administration of a therapeutically effective amount of an iodiscorbate and pharmaceutically acceptable salts thereof to a subject in need thereof.

Therefore, the compounds of the present inventions are useful in the inhibition, prevention or treatment of infection by human immunodeficiency virus HIV and for treating consequent pathological conditions such as AIDS. Treating AIDS or preventing or treating infection by HIV is defined as including, but not limited to, treating a wide range of states of HIV infection: AIDS, ARC (AIDS related complex), both symptomatic and asymptomatic, and actual or potential exposure to HIV. For example, the compounds of this invention are useful in treating infection by HIV after suspected past exposure to HIV by e.g., blood transfusion, exchange of body fluids, bites, accidental needle stick, or exposure to patient blood during surgery.

In any of the above-identified disease states or conditions, the iodiscorbate has a general formula of XISrC₆H₅O₆, wherein X can be either bismuth, potassium, or zinc. Consequently, useful iodiscorbates of the invention include, for example, BiISrC₆H₅O₆, K₂₁SrC₆H₅O₆, and ZnISrC₆H₅O₆. Additionally, the iodiscorbate is considered as a compound that includes operably bonded iodine, ascorbic acid, strontium, and either bismuth, potassium, or zinc.

Each of the above-identified treatments provided by the present invention provide for the inhibition and/or the prevention viral reverse transcription, viral post-reverse transcription, viral integration, and/or viral post-integration. In another aspect, the subject that utilizes the advantageous administration of the therapeutics iodiscorbate compounds of the invention is a human.

The method of the invention can further include administration of protease inhibitors, nucleoside analog reverse transcriptase inhibitors and non-nucleoside reverse transcriptase inhibitors. Suitable protease inhibitors include, for example, saquinavir, ritonavir, indinavir, nelfinavir, and amprenavir. Nucleoside analog reverse transcriptase inhibitors include, for example, zidovudine, didanosine, zalcitabine, stavudine, lamivudine, and abacavir. Suitable non-nucleoside reverse transcriptase inhibitors include, for example, nevirapine, efavirenz, and delavirdine.

The invention further pertains to packaged pharmaceutical compositions useful for the above-identified disease states or conditions, i.e., treating human immunodeficiency virus (HIV), acquired immunodeficiency syndrome (AIDS), AIDS related complex (ARC), or AIDS related diseases in a subject. The packaged pharmaceuticals include a container holding a therapeutically effective amount of an iodiscorbate and pharmaceutically acceptable salts thereof, as described herein, and appropriate instructions for using the iodiscorbate for treating the particular condition in the subject.

The packaged pharmaceuticals can further include protease inhibitors, nucleoside analog reverse transcriptase inhibitors and non-nucleoside reverse transcriptase inhibitors. The protease inhibitors, nucleoside analog reverse transcriptase inhibitors and non-nucleoside reverse transcriptase inhibitors include those identified throughout this application; however, these are identified as examples only and should not be construed as limiting.

The present invention is also directed to combinations of the HIV integrase inhibitor compounds with one or more agents useful in the treatment of AIDS. For example, the compounds of this invention may be effectively administered, whether at periods of pre-exposure and/or post-exposure, in combination with effective amounts of the AIDS antivirals, imunomodulators, antiinfectives, or vaccines, such as those including the following. Suitable antivirals, for example include drug 097 (Hoechst/Bayer) suitable for HIV infection, AIDS, or ARC as a non-nucleoside reverse transcriptase (RT) inhibitor; drug 141 W94 (Glaxo Wellcome) suitable for HIV infection, AIDS, or ARC as a protease inhibitor; drug 1592U89 (Glaxo Wellcome) suitable for HIV infection, AIDS, or ARC as a protease inhibitor; Abacavir (1592U89) (Glaxo Wellcome) suitable for HIV infection, AIDS, or ARC as a RT inhibitor; Acmannan (Carrington Labs) suitable for ARC; Acyclovir (Burroughs Wellcome) suitable for HIV infection, AIDS, or ARC, in combination with AZT; AD-439 (Tanox Biosystems) suitable for HIV infection, AIDS, or ARC; AD-519 (Tanox Biosystems) suitable for HIV infection, AIDS, or ARC; Adefovir dipivoxil (Gilead Sciences) suitable for HIV infection; AL-721 (Ethigen) suitable for ARC; Alpha Interferon (Glaxo Wellcome) suitable for Kaposi's sarcoma, HIV in combination with Retrovir; Ansamycin (Adria Laboratories) suitable for ARC; LM 427 (Erbamont); AR177 (Aronex Pharm) suitable for HIV infection, AIDS, or ARC; beta-fluoro-ddA (Nat'l Cancer Institute) suitable for AIDS-associated diseases; BMS-232623 (Bristol-Myers Squibb) suitable for HIV infection or AIDS; CGP-73547 (Novartis) suitable for ARC as a protease inhibitor; BMS-234475 (Bristol-Myers Squibb) suitable for HIV infection or AIDS; CGP-61755 (Novartis) suitable for ARC as a protease inhibitor; CI-1012 (Warner-Lambert) suitable for HIV-1 infection; Cidofovir (Gilead Science) suitable for CMV retinitis, herpes, papillomavirus; Curdlan sulfate (AJI Pharma USA) suitable for HW infection; Cytomegalovirus immune globin (MedImmune) suitable for CMV retinitis; Cytovene (Syntex) suitable for sight threatening CMV; Ganciclovir suitable for peripheral CMV retinitis; Delaviridine (Pharmacia-Upjohn) suitable for HIV infection, AIDS, or ARC as a RT inhibitor; Dextran Sulfate (Ueno Fine Chem. Ind. Ltd. Osaka, Japan) suitable for AIDS, ARC, or HIV; ddC (Dideoxycytidine) (Hoffman-La Roche) suitable for HW infection, AIDS or ARC; ddI (Dideoxycytidine) suitable for ARC; ddI (Dideoxyinosine) (Bristol-Myers Squibb) suitable for HIV infection, AIDS, or ARC; DMP-450 (AVID) suitable for HIV infection, AIDS, or ARC as a protease inhibitor; Efavirenz (DuPont Merck) suitable for HIV infection, AIDS, or ARC as a non-nucleoside RT inhibitor; STOCRINE EL10 (Elan Corp, PLC) suitable for HIV; Famciclovir (Smith Kline) suitable for herpes zoster or herpes simplex; FTC (Emory University) suitable for HIV infection, AIDS, or ARC as a reverse transcriptase inhibitor; GS 840 (Gilead) suitable for HIV infection, AIDS, or ARC as a reverse transcriptase inhibitor; GW 141 (Glaxo Welcome) suitable for HIV infection, AIDS, or ARC as a protease inhibitor; GW 1592 (Glaxo Welcome) suitable for HIV infection, AIDS, or ARC as a reverse transcriptase inhibitor); HBY097 (Hoechst Marion Roussel) suitable for HIV infection, AIDS, or ARC as a non-nucleoside reverse transcriptase inhibitor; Hypericin (VIMRX Pharm.) suitable for HIV infection, AIDS, or ARC; Recombinant Human Interferon Beta (Triton Biosciences) suitable for AIDS, Kaposi's sarcoma, or ARC; Interferon alfa n-3 (Interferon Sciences) suitable for ARC or AIDS; Indinavir (Merck) suitable for HIV infection, AIDS, ARC, or asymptomatic HIV positive, also in combination with AZT/ddI/ddC; ISIS 2922 (ISIS Pharmaceuticals) suitable for CMV retinitis; KNI-272 (Nat'l Cancer Institute) suitable for HIV diseases; Lamivudine, 3TC (Glaxo Wellcome) suitable for HIV infection, AIDS, or ARC as a reverse transcriptase inhibitor, also in combination with AZT; Lobucavir (Bristol-Myers Squibb) suitable for CMV infection; Nelfinavir (Agouron Pharmaceuticals) suitable for HIV infection, AIDS, or ARC as a protease inhibitor; Nevirapine (Boeheringer Ingleheim) suitable for HIV infection, AIDS, or ARC as a RT inhibitor); Novapren (Novaferon Labs, Inc.) suitable for HIV inhibitor; Peptide T Octabpeptide Sequence (Peninsula Labs) suitable for AIDS; Trisodium Phosphonoformate (Astra Pharm. Products, Inc.) suitable for CMV retinitis, HIV infection, and other CMV infections; PNU-140690 (Pharmacia Upjohn) suitable for HIV infection, AIDS, or ARC as a protease inhibitor; Probucol (Vyrex) suitable for HIV infection, or AIDS; RBC-CD4 (Sheffield Med. Tech) suitable for HIV infection, AIDS, or ARC; Ritonavir (Abbott) suitable for HIV infection, AIDS, or ARC as a protease inhibitor; Saquinavir (Hoffmann-LaRoche) suitable for HIV infection, AIDS, or ARC as a protease inhibitor; Stavudine (Bristol-Myers Squibb) suitable for HIV infection, AIDS, or ARC; Valaciclovir (Glaxo Wellcome) suitable for genital HSV & CMV infections; Virazole Ribavirin (Viratek/ICN) suitable for asymptomatic HIV; VX-478 (Vertex) suitable for HIV infection, AIDS, or ARC; Zalcitabine (Hoffmann-La Roche) suitable for HIV infection, AIDS, or ARC, with AZT; Zidovudine; AZT (Glaxo Wellcome) suitable for HIV infection, AIDS, ARC, or Kaposi's sarcoma. Immunomodulators include AS-101 (Wyeth-Ayerst) suitable for AIDS; Bropirimine (Pharmacia Upjohn) suitable for advanced AIDS; Acemannan (Carrington Labs, Inc.) suitable for AIDS or ARC; CL246,738 (American Cyanamid Lederle Labs) suitable for AIDS or Kaposi's sarcoma; EL10 (Elan Corp, PLC) suitable for HIV infection; FP-21399 (Fuki ImmunoPharm) suitable for blocking HIV fusion with CD4 cells; Gamma Interferon (Genentech) suitable for ARC, in combination with TNF (tumor necrosis factor); Granulocyte Macrophage Colony Stimulating Factor (Genetics Institute Sandoz) suitable for AIDS; IL-2 (Cetus) suitable for AIDS, Immune Globulin Intravenous (Cutter Biological) suitable for pediatric AIDS, in combination w/AZT; IMREG-1 (Imreg) suitable for AIDS, Kaposi's sarcoma, ARC, or PGL; IMREG-2 (Imreg) suitable for AIDS, Kaposi's sarcoma, ARC, or PGL; Imuthiol Diethyl Dithio Carbamate (Merieux Institute) suitable for AIDS, or ARC; Alpha-2 Interferon (Schering Plough) suitable for Kaposi's sarcoma or AIDS in combination with AZT; Methionine-Enkephalin (TNI Pharmaceutical) suitable for AIDS, or ARC; MTP-PE (Ciba-Geigy Corp.) suitable for Kaposi's sarcoma; Granulocyte Colony Stimulating Factor (Amgen) suitable for AIDS, in combination with AZT; Remune (Immune Response Corp.) suitable as a immunotherapeutic; rCD4 (Genentech) suitable for AIDS, or ARC; Recombinant Soluble Human CD4 (Biogen) suitable for AIDS or ARC; Interferon Alfa-2 (Hoffman-La Roche) suitable for Kaposi's sarcoma AIDS, or ARC, in combination with AZT; SK&F106528 (Smith Kline) suitable for HIV infection; Soluble T4 Thymopentin (Immunobiology Research Institute) suitable for HIV infection; Tumor Necrosis Factor (TNF) (Genentech) suitable for ARC, in combination with gamma Interferon. Anti-infectives include, for example, Clindamycin with Primaquine (Pharmacia Upjohn) suitable for PCP; Fluconazole (Pfizer) suitable for cryptococcal meningitis or candidiasis; Pastille (Squibb Corp.) suitable for the prevention of oral candidiasis; Ornidyl (Merrell Dow) suitable for PCP; Eflornithine Pentamidine (LyphoMed) suitable for PCP treatment; Spiramycin (Rhone-Poulenc) suitable for cryptospoildial diarrhea; Intraconazole-R51211 (Janssen Pharm.) suitable for histoplasmosis or cryptococcal meningitis; and trimetrexate (Warner-Lambert) suitable for PCP. Other drugs that fall within several of the above-identified categories, include, for example, Daunorubicin (NeXstar, Sequus) suitable for Karposi's sarcoma; Recombinant Human Erythropoietin (Ortho Pharmaceutical Corp.) suitable for severe anemia associated with AZT therapy; Recombinant Human Growth Hormone (Serono) suitable for AIDS-related wasting or cachexia; Megestrol Acetate (Bristol-Myers Squibb) suitable for the treatment of anorexia associated with AIDS; Testosterone (Alza, Smith Kline) suitable for AIDS-related wasting; Total Enteral Nutrition (Norwich Eaton Pharmaceuticals) suitable for diarrhea and malabsorption related to AIDS.

The terms “treat”, “treating” or “treatment” are intended to include the ability of the iodiscorbate therapeutic compounds of the invention to inhibit, prevent, ameliorate, or, eradicate the disease state or condition associated identified as causing the identified affliction in the subject.

The phrase “disease state or condition” is intended to include those symptoms commonly recognized as being associated with the particular ailment. Disease states or conditions, as used herein, refer to those ailments and/or afflictions associated with human immunodeficiency virus (HIV), acquired immunodeficiency syndrome (AIDS), AIDS related complex (ARC), or AIDS related diseases. These terms are art recognized and therefore, one skilled in the art would recognize the various possible disease states or conditions that are related to such ailments or afflictions. Such ailments or afflictions include, but are not limited to, such disease states or conditions identified as related to AIDS.

The phrase “human immunodeficiency virus” (HIV) is recognized in the art and is intended to refer to a retrovirus which is the etiological agent of the complex disease that includes progressive destruction of the immune system (acquired immune deficiency syndrome; “AIDS”) and degeneration of the central and peripheral nervous system. This virus was previously known as LAV, HTLV-III, or ARV. A common feature of retrovirus replication is the insertion by virally-encoded integrase of proviral DNA into the host cell genome, a required step in HIV replication in human T-lymphoid and monocytoid cells. Integration is believed to be mediated by integrase in three steps: assembly of a stable nucleoprotein complex with viral DNA sequences; cleavage of two nucleotides from the 3′ termini of the linear proviral DNA; covalent joining of the recessed 3′ OH termini of the proviral DNA at a staggered cut made at the host target site. The fourth step in the process, repair synthesis of the resultant gap, may be accomplished by cellular enzymes.

The phrase “acquired immune deficiency syndrome” (AIDS) is recognized in the art and is intended to include, but not be limited to a defect in cell-mediated immunity, occurring in a person with no known cause for diminished resistance to that disease. Such diseases include Kaposi's sarcoma (KS), PCP Pneumocystis carinii pneumonia (PCP), and serious other opportunistic infections (001). These infections include pneumonia, meningitis, or encephalitis due to one or more of the following: aspergillosis, candidiasis, cryptococcosis, cytomegalovirus, norcardiosis, strongyloidosis, toxoplasmosis, zygomycosis, or a typical mycobacteriosis (species other than tuberculosis or lepra); esophagitis due to candidiasis, cytomegalovirus, or herpes simplex virus; progressive multifocal leukoencephalopathy, chronic enterocolitis (more than 4 weeks) due to cryptosporidiosis; or unusually extensive mucocutancous herpes simplex of more than 5 weeks duration. Diagnoses are considered to fit the case definition only if based on sufficiently reliable methods (generally histology or culture). However, this case definition may not include the full spectrum of AIDS manifestations, which may range from absence of symptoms (despite laboratory evidence of immune deficiency) to nonspecific symptoms (e.g., fever, weight loss, generalized, persistent lymphadenopathy) to specific diseases that are insufficiently predictive of cellular immunodeficiency to be included in incidence monitoring (e.g., tuberculosis, oral candidiasis, herpes zoster) to malignant neoplasms that cause, as well as result from, immunodeficiency.(

The phrase “AIDS related complex”, (ARC) is recognized in the art and is intended to include reference to the symptomatic HIV infection. It is an older term used to describe a condition in which an HIV positive person has a variety of symptoms related to HIV disease (e.g., swollen lymph nodes, night sweats, fever, diarrhea) that do not qualify as AIDS-defining illnesses. The symptoms of ARC are typically less severe than those of full-blown AIDS.

The phrase “AIDS related diseases” as used herein is recognized in the art and is intended to include one or more indicator diseases (opportunistic infections) including, but not limited to, Kaposi's sarcoma, invasive cervical cancer, pneumocystis carinii pneumonia, and wasting syndrome.

The phrase “antiviral target” as used herein refers to one stage of the HIV lifecycle at which the present invention works to inhibit, prevent, ameliorate, or eradicate the virus.

The phrase “reverse transcription” is recognized in the art and is intended to include the stage in the virus lifecycle were the virion enzyme in HIV, reverse transcriptase, catalyzes the conversion of viral RNA into single-stranded DNA.

The phrase “post reverse transcription” as used herein is recognized in the art and means the stage in the virus lifecycle between reverse transcription and integration.

The term “integration” as used herein is recognized in the art and is intended to include the stage of the virus lifecycle when the viral DNA enters the host cell nucleus where the enzyme integrase catalyzes its integration into the genetic material of the cell.

The phrase “post integration” as used herein is recognized in the art and is intended to refer to the stage in the virus lifecycle between integration and the breakdown of proteins by protease.

The term “iodiscorbate” is intended to include those therapeutic compounds formed by the combination of an ascorbic acid, a heavy metal salt such as strontium iodide (SrI), and a diamagnetic ion, such as a salt. Particular compounds feature iodine bonded to a stabilized metal salt (such as bismuth, zinc, or potassium) which is bonded to strontium and an organic acid (ascorbic acid). Thus, each of the compounds of the invention has four components: a diamagnetic ion (such as bismuth, zinc, or potassium), iodine, strontium, and ascorbic acid. The diamagnetic ion (bismuth being the most diamagnetic of all the diamagnetic elements) serves to direct the compound to interfere with the viral replication process.

The term “pharmaceutically acceptable salts, or prodrugs” as used herein refers to those salts, carboxylic acid addition salts, and prodrugs of the iodiscorbates of the present invention which are, within the scope of sound medical judgment, suitable for use in contact with the tissues of patients without undue toxicity, irritation, allergic response, and the like, commensurate with a reasonable benefit/risk ratio, and effective for their intended use of the compounds of the invention. The term “salts” refers to the relatively non-toxic, inorganic and organic acid addition salts of compounds of the present invention. These salts can be prepared in situ during the final isolation and purification of the compounds or by separately reacting the purified compound in, for example, its acid form with a suitable bases and isolating the salt thus formed. These can include cations based on the alkali and alkaline earth metals, such as sodium, lithium, potassium, calcium, magnesium and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See, for example, Berge S. M., et al., “Pharmaceutical Salts,” J. Pharm. Sci., 1977;66:1-19 which is incorporated herein by reference).

The term “prodrug” refers to compounds that are rapidly transformed in vivo to yield iodiscorbate analogues by hydrolysis in blood. A thorough discussion is provided in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference. As used herein, a prodrug is a compound that, upon in vivo administration, is metabolized or otherwise converted to the biologically, pharmaceutically or therapeutically active form of the compound. To produce a prodrug, the pharmaceutically active compound, the iodiscorbate, is modified such that the active compound will be regenerated by metabolic processes. The prodrug can be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, once a pharmaceutically active compound is identified, those of skill in the pharmaceutical art generally can design prodrugs of the compound (See, e.g., Nogrady (1985) Medicinal Chemistry A Biochemical Approach, Oxford University Press, New York, pages 388-392). Conventional procedures for the selection and preparation of suitable prodrug derivatives are described, for example, in “Design of Prodrugs,” ed. H. Bundgaard, Elsevier, 1985.

The term “subject” as used herein refers to any living organism in which a HIV, AIDS, ARC or AIDS related disease response is elicited. The term subject includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

The term “mammal” as used herein refers to a living organism capable of eliciting a response to the treatment of AIDS, HIV, ARC or AIDS related diseases. The term subject includes, but is not limited to, humans, nonhuman primates such as chimpanzees and other apes and monkey species, sheep, pigs, goats, horses, dogs, cats, mice, rats and guinea pigs, and the like.

The pharmaceutical compositions of the invention described herein include a “therapeutically effective amount” or a “prophylactically effective amount” of the iodiscorbates of the invention. A “therapeutically effective amount” refers to an amount effective, at dosages and for periods of time necessary, to achieve the desired therapeutic result, e.g., an eradication, diminishment, inhibition, or prevention of HIV, AIDS, ARC or AIDS related disease, from the subject, at a reasonable benefit/risk ratio applicable to any medical treatment.

A therapeutically effective amount of the iodiscorbates of the invention vary according to factors such as the disease state, age, sex, and weight of the individual, and the ability of the iodiscorbate to elicit a desired response in the individual. A therapeutically effective amount is also one in which any toxic or detrimental effects of HIV, AIDS, ARC or AIDS related disease are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease state or condition, the prophylactically effective amount will generally be less than the therapeutically effective amount.

For example, the iodiscorbate is administered at a therapeutically effective dosage sufficient to inhibit, eradicate, prevent or diminish, one or more disease states or conditions associated with HIV, AIDS, ARC or AIDS related diseases. A “therapeutically effective dosage” reduces the disease state or condition in the subject by at least about 20%, more particularly by at least about 40%, even more particularly by at least about 60%, and still more particularly by at least about 80% to 90% relative to untreated subjects. The ability of a compound to inhibit or ameliorate HIV, AIDS, ARC or AIDS related disease mediated responses can be evaluated in an animal model system that can be predictive of efficacy in treating said responses. Monitoring can be performed by well known quantitative analytical techniques that can identify the reduction and/or eradication of the virus and related components.

The present invention provides pharmaceutically acceptable compositions which comprise a therapeutically-effective amount of one or more of the iodiscorbate(s) described above, formulated together with one or more pharmaceutically acceptable carriers (additives) and/or diluents. As described in detail below, the pharmaceutical compositions of the present invention can be specially formulated for administration in solid or liquid form, including those adapted for the following: oral administration, for example, drenches (aqueous or non-aqueous solutions or suspensions), tablets, boluses, powders, granules, pastes for application to the tongue; parenteral administration, for example, by subcutaneous, intramuscular or intravenous injection as, for example, a sterile solution or suspension; topical application, for example, as a cream, ointment or spray applied to the skin; as an aerosol, or intravaginally or intrarectally, for example, as a pessary, cream or foam.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the iodiscorbate from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. As set out above, the iodiscorbates of the present invention can contain at least one acidic functional group and, thus, is capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically-acceptable metal action, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the pharmaceutical compositions.

Examples of pharmaceutically-acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like.

Formulations of the present invention include those suitable for oral, nasal, topical (including buccal and sublingual), rectal and/or parenteral administration. The formulations can conveniently be presented in unit dosage form and can be prepared by any methods well known in the art of pharmacy. The amount of iodiscorbate which can be combined with a carrier material to produce a single dosage form will vary depending upon the subject being treated, and/or the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, in particular from about 5 percent to about 70 percent, most particularly from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration can be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as bronchoaveolar lavages for intended delivery systems to the lung and the like, each containing a predetermined amount of an iodiscorbate of the present invention as an active ingredient. An iodiscorbate of the present invention can also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof, and coloring agents. In the case of capsules, tablets and pills, the pharmaceutical compositions can also comprise buffering agents. Solid compositions of a similar type can also be employed as fillers in soft and hard-filled gelatin capsules using such excipients as lactose or milk sugars, as well as high molecular weight polyethylene glycols and the like.

A tablet can be made by compression or molding, optionally with one or more accessory ingredients. Compressed tablets can be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets can be made by molding in a suitable machine a mixture of the powdered compound moistened with an inert liquid diluent.

The tablets, and other solid dosage forms of the pharmaceutical compositions of the present invention, such as dragees, capsules, pills and granules, can optionally be scored or prepared with coatings and shells, such as enteric coatings and other coatings well known in the pharmaceutical-formulating art. They can also be formulated so as to provide slow or controlled release of the active ingredient therein using, for example, hydroxypropylmethyl cellulose in varying proportions to provide the desired release profile, other polymer matrices, liposomes and/or microspheres. They can be sterilized by, for example, filtration through a bacteria-retaining filter, or by incorporating sterilizing agents in the form of sterile solid compositions which can be dissolved in sterile water, or some other sterile injectable medium immediately before use. These compositions can also optionally contain opacifying agents and can be of a composition that they release the active ingredient(s) only, or in certain aspects, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the iodiscorbates of the invention include pharmaceutically acceptable emulsions, microemulsions, solutions, suspensions, syrups and elixirs. In addition to the active ingredient, the liquid dosage forms can contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming and preservative agents.

Suspensions, in addition to the iodiscorbate, can contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration can be presented as a suppository, which can be prepared by mixing one or more iodiscorbates of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum and release the active compound.

Formulations of the present invention which are suitable for vaginal administration also include pessaries, creams, gels, pastes, foams or spray formulations containing such carriers as are known in the art to be appropriate.

Dosage forms for the topical or transdermal administration of iodiscorbates of this invention include powders, sprays, ointments, pastes, creams, lotions, gels, solutions, patches and inhalants. The iodiscorbate can be mixed under sterile conditions with a pharmaceutically-acceptable carrier, and with any preservatives, buffers, or propellants which can be required.

The ointments, pastes, creams and gels can contain, in addition to an iodiscorbate of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an iodiscorbate of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane. The iodiscorbate of the invention can be administered topically within the airways, e.g. by the pulmonary route/by inhalation. Advantageously, while having potent efficacy when administered topically, the iodiscorbates of the invention are devoid of, or exhibit relatively reduced, systemic activity, e.g. following oral administration. The iodiscorbates of the invention thus provide a means for the treatment of diseases and conditions as hereinabove set forth, with the avoidance of unwanted systemic side effect, e.g. consequent to inadvertent swallowing of drug substance during inhalation therapy.

Transdermal patches have the added advantage of providing controlled delivery of an iodiscorbate of the present invention to the body. Such dosage forms can be made by dissolving or dispersing the iodiscorbate in the proper medium. Absorption enhancers can also be used to increase the flux of the iodiscorbate across the skin. The rate of such flux can be controlled by either providing a rate controlling membrane or dispersing the iodiscorbate in a polymer matrix or gel.

Ophthalmic formulations, eye ointments, powders, solutions and the like, are also contemplated as being within the scope of this invention.

Pharmaceutical compositions of this invention suitable for parenteral administration comprise one or more iodiscorbates of the invention in combination with one or more pharmaceutically-acceptable sterile isotonic aqueous or nonaqueous solutions, dispersions, suspensions or emulsions, or sterile powders which can be reconstituted into sterile injectable solutions or dispersions just prior to use, which can contain antioxidants, buffers, solutes which render the formulation isotonic with the blood of the intended recipient or suspending or thickening agents.

Examples of suitable aqueous and nonaqueous carriers which can be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

The pharmaceutical compositions can also contain adjuvants such as preservatives, wetting agents, emulsifying agents and dispersing agents. Prevention of the action of microorganisms can be ensured by the inclusion of various antibacterial and antifungal agents, for example, paraben, chlorobutanol, phenol sorbic acid, and the like. It is also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin.

In some cases, in order to prolong the effect of an iodiscorbate, it is desirable to slow the absorption of the iodiscorbate from subcutaneous or intramuscular injection. This can be accomplished by the use of a liquid suspension of crystalline or amorphous material having poor water solubility. The rate of absorption of the iodiscorbate then depends upon its rate of dissolution which, in turn, can depend upon crystal size and crystalline form. Alternatively, delayed absorption of a parenterally-administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the therapeutic agent in liposomes or microemulsions which are compatible with body tissue.

When the iodiscorbates of the present invention are administered as pharmaceuticals, to humans and animals, they can be given per se or as a pharmaceutical composition containing, for example, 0.1 to 99.5% (more particularly, 0.5 to 90%) of active ingredient in combination with a pharmaceutically acceptable carrier.

The preparations of the present invention can be given orally, parenterally, topically, or rectally. They are of course given by forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administration is often most easily employed.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrastemal injection and infusion.

The phrases “systemic administration,” “administered systemically,” “peripheral administration” and “administered peripherally” as used herein mean the administration of an iodiscorbate other than directly into the central nervous system, such that it enters the patient's system and, thus, is subject to metabolism and other like processes, for example, subcutaneous administration.

Regardless of the route of administration selected, the iodiscorbates of the present invention, which can be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention can be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of an iodiscorbate of the invention will be that amount of the compound which is the lowest dose effective to produce a therapeutic effect. Such an effective dose will generally depend upon the factors described above. Generally, intravenous, intracerebroventricular and subcutaneous doses of the compounds of this invention for a patient, when used for the indicated analgesic effects, will range from about 0.0001 to about 100 mg per kilogram of body weight per day, more particularly from about 0.01 to about 50 mg per kg per day, and still more particularly from about 0.1 to about 40 mg per kg per day.

If desired, the effective daily dose of the iodiscorbate can be administered as two, three, four, five, six or more sub-doses administered separately at appropriate intervals throughout the day, optionally, in unit dosage forms.

While it is possible for a compound of the present invention to be administered alone, it is possible to administer the compound as a pharmaceutical composition.

In other embodiments, the iodiscorbate can be administered in combination with either a protease inhibitor, a nucleoside analog or a non-nucleoside analog as described above.

The present invention is therefore directed toward (1) compounds designed to inhibit and eradicate HIV, AIDS, ARC, and AIDS related diseases, and (2) to identifying methods for using these compounds. The compounds of the invention feature iodine bonded to a stabilized metal salt (such as bismuth, potassium, or zinc) which is bonded to strontium and an organic acid (ascorbic acid). Thus, each of the compounds of the invention has four components: a diamagnetic ion (such as potassium or zinc), iodine, strontium, and ascorbic acid.

The preparation of the compounds of the invention now will be described. There are many methods for synthesizing the compounds of the present invention. Each is acceptable and within the scope of the present invention. The bismuth compound may be prepared from SrI, bismuth, and D- or L-ascorbic acid in mole ratios of 1:1. The ascorbic acid is dissolved in glycerol (CHOH(CH₂OH)₂) to distribute the ascorbic acid molecules symmetrically, thus exposing the ascorbic acid's reactive hydroxyl sites. SrI, a subhalide of strontium, then is added in an amount that is not sufficient to neutralize the acid. Next, an aqueous solution of Bi(NO₃)₃ is added in an amount that is approximately equivalent to that of the ascorbic acid. Additional SrI then is added to raise the pH above 8.0. Finally, ascorbic acid is added to lower the solution's pH to a level acceptable for therapeutic use.

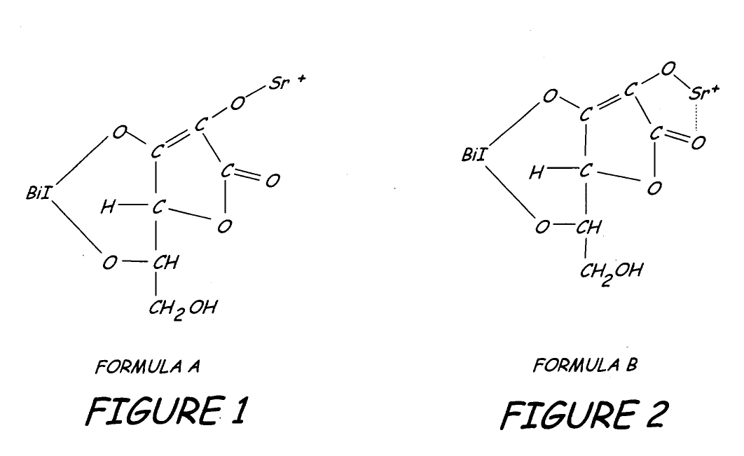

The compound depicted as Formula A (FIG. 1) has the formula BiISrC₆H₅O₆, and can be produced by performing the above-described process at a temperature of about 25° C. By way of specific example, the compound may be produced as follows: Equal molar quantities of D- or L-ascorbic acid, SrI, and Bi(NO₃)₃ are added at a temperature of 25° C. First, 176 g of ascorbic acid are dissolved in 500 ml of glycerol. Next, 100 g of SrI (an amount insufficient to neutralize the acid) is added while the solution is stirred and cooled to 25° C. Next, 20% increments of 500 ml aqueous solution containing 395 g of Bi(NO₃)₃ are added alternately with 20% increments of 114 g of SrI while the solution is stirred and maintained at a temperature of 25° C. An orange salt will precipitate out of the solution. This precipitate is centrifuged and washed several times with distilled water by centrifugation. Next, 5 g of the moist precipitate is suspended in 100 ml of 50% glycol. If the solution is cloudy, it may be filtered, and a stabilizing solution of 2.5 cc Na₂SO₃ solution (containing 250 mg Na₂SO₃) may be added. The compound is rendered suitable for therapeutic use by adding additional ascorbic acid to lower the pH to about 7.6. The compound that is produced by this method may be water soluble or insoluble.

The compound depicted as Formula B (FIG. 2) has the formula BiISrC₆H₅O₆ and has a stereochemical conformation (i.e., the intermolecular hydrogen bond and its ionic association with the univalent strontium are in mobile equilibrium). Formula B is prepared in the same manner as is Formula A, except upon the initial addition of SrI the solution is rapidly cooled to and maintained at 15° C.

It is to be noted that SrI is a subhalide of strontium, and is obtained from the free metal and the normal halide by heating SrI₂ and strontium to a temperature of at least 780° C. This melt is chilled quickly to room temperature to avoid the possibility of reversion at the intermediate temperatures. It is to be noted that the subhalide, SrI, decomposes in time under normal conditions into the free metal and the normal halide. SrI is an intense brown, well-crystallized, and hygroscopic. When decomposed, SrI forms Sr(OH)₂ and ordinary halide. The bismuth should be used in the form of bismuth nitrate. The nitrate acts as a catalyst and as an oxidizing agent to enhance direct iodination, resulting in the compound's iodination product.

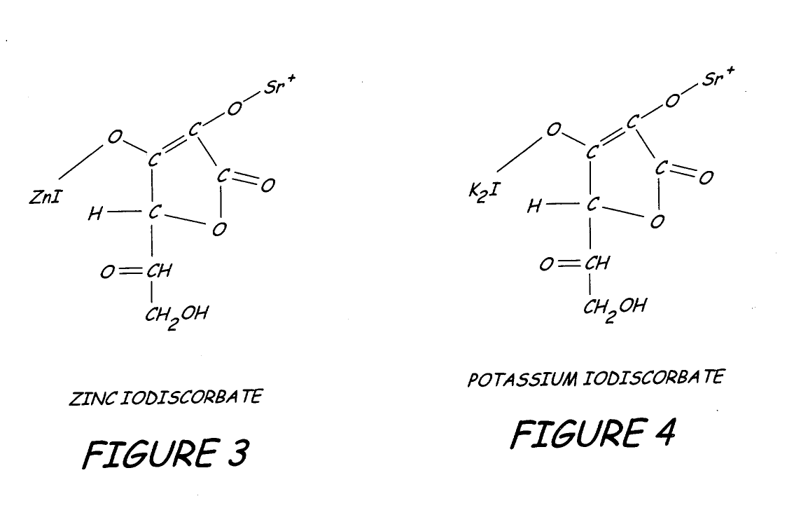

ZnSrI ascorbate, entitled zinc iodiscorbate, may be synthesized as follows: Zinc oxide and strontium hydroxide are added in 1:1 molar concentrations to deionized water. Hydriodic acid (1 mole, 57 weight percent) is stirred in (a suspension still will be present after several hours). Ascorbic acid (3 moles) is added; this dissolves the suspension, resulting in a clear solution. The water is removed under vacuum at a temperature of approximately 25° C., leaving behind a crystalline solid. The empirical formula for the zinc iodiscorbate is ZnSrI(ascorbate)₃. The potassium iodiscorbate is synthesized in a similar manner, substituting potassium for zinc (refer to FIGS. 3 and 4). This compound produced by this method also may be water soluble or insoluble. For the compounds of the present invention, solubility in water is advisable because the compounds may be easier to work with.

The following are additional and more simplified methods for synthesizing three of the compounds of the invention:

### Bismuth Iodiscorbate

Bismuth iodiscorbate was prepared by dissolving 58 g of concentrated hydriodic acid (56.1 weight-percent) into 350 mls of water. Next, 5 g of 0.0085 molar bismuth (III) iodide was crushed in a mortar, and then dissolved in the solution. Next, 2.3 g of 0.0085 molar strontium hydroxide octahydrate was stirred into the solution, followed by the addition of 4.5 g of 0.026 molar L-ascorbic acid. The mixture was stirred for three hours, then stripped to dryness on a rotary evaporator. The residue then was redissolved in 500 ml water and filtered, and again stripped on a rotary evaporator. This process assisted in the removal of excess hydriodic acid. The residue again was dissolved in 500 ml of water, filtered, and stripped to dryness on a rotary evaporator. An orange solid then was removed from the flask and bottled. This procedure yielded 9.1 g of what is designated as SrBiI₂[ascorbate]₃. This compound was water soluble.

### Potassium Iodiscorbate

Potassium iodiscorbate was prepared by adding 6.5 g of 0.12 molar potassium hydroxide and 16.0 g of 0.06 molar strontium hydroxide octahydrate to 500 ml water in a 1000-ml beaker, resulting in a white, cloudy solution. The mixture was stirred for about 2 hours. Next, 13.0 g of 0.06 molar hydriodic acid (56 weight-percent) was added, and the mixture was stirred for three hours. Next, 3.2 g of 0.18 molar L-ascorbic acid was added, turning the mixture clear in about 5 minutes. The mixture then was stirred for another 0.5 hour. The water was removed under vacuo, and the residue dried under vacuum for about 8 hours. The solids were removed from the flask and bottles. The structure is designated as [ascorbate]₂SrK[ascorbate]KI. This compound also was water soluble.

### Zinc Iodiscorbate

Zinc iodiscorbate was prepared by adding 4.0 g of 0.05 molar zinc oxide and 13.3 g of 0.05 molar strontium hydroxide octahydrate to 500 ml of distilled water, resulting in a white suspension. The mixture then was treated with 11.2 g of 0.05 molar concentrated hydriodic acid and stirred for two hours. A white suspension still remained. Next, 26.4 g of 0.15 molar L-ascorbic acid was added, and the reaction mixture became colorless within 20 minutes. The solution was evaporated to dryness on a rotary evaporator, then dried under vacuo for 8 hours at room temperature. The resulting yellow-orange powder was transferred to a tarred bottle. The structure of this compound was designated [ascorbate]₂SrZn[ascorbate]I. This compound also was water soluble.

The following experiments demonstrate the ability of iodiscorbate compounds of the invention to inhibit the replication of Human Immunodeficiency Virus (HIV), the virus that causes AIDS. (Compounds B, K and Z relate respectively to the Bismuth, Potassium and Zinc iodiscorbates) The compounds were good inhibitors of HIV virus replication in cultured human blood cells, and also had activity against latently infected cells. This was very encouraging, since current AIDS therapies have not been able to eliminate the reservoir(s) of latently infected cells responsible for virus re-bound and disease progression after patients discontinue or become resistant to their treatments.

### Methods

PBMC Isolation and Blasting

Peripheral blood monocular cells (PBMCs) were obtained from normal hepatitis B and C and HIV-1 negative donors by ficoll hypaque gradient separation. Briefly, anti-coagulated blood was diluted 1:1 with Dulbecco's phosphate buffered saline without Ca⁺² and Mg^{+@} (PBS) and layered over 14 mL of Lymphocyte separation media in a 50 ml centrifuge tube. Tubes were then centrifuged for 30 minutes at 600×g. Banded PBLs were gently aspirated from the resulting interface and subsequently washed 2× with PBS by low speed centrifugation. The mononuclear cells were counted, viability determined by Trypan Blue dye exclusion and resuspended in RPMI 1640 medium supplemented with 15% FBS (heat inactivated), 2 mM L-glutamine, 100 U/mL penicillin, 100 μg/mL streptomycin, and 10 μg/mL gentamycin with 2 μg/mL phytohemagluttin (PHA) at 1×10⁶ cells/mL. The cells were cultured for 48 to 72 h at 37° C., 5%, C⁰². Following incubation, cells were collected by centrifugation, washed and resuspended in RPMI 1640 supplemented with 15% FBS (heat inactivated), 2 mM L-glutamine, 100 U/mL penicillin, 100 μg/mL streptomycin, and 10 μg/mL gentamycin with 40 U/mL, recombinant IL-2 (R & D Systems, Minneapolis, Minn.). IL-2 was included in the culture medium to maintain the cell division initiated by the PHA mitogenic stimulation. The cultures were then maintained until use by ½ culture volume change with fresh IL-2 containing medium every 3 days.

PBMC Assay

Human peripheral blood mononuclear cells from a minimum of 2 donors, that were blasted with PHA and IL-2, were counted, viability determined by Trypan Blue dye exclusion and mixed in equal ratios. Pooled donors were used to minimize the variability observed between individual donors which results from quantitative and qualitative differences in HIV infection and overall response to the PHA and IL-2 of primary lymphocyte populations. The cells were resuspended at 1×10⁶ cells/mL in RPM1 1640 without phenol red supplemented with 15% Fetal Bovine Serum (heat inactivated), 2 nM L-glutamine, 100 U/mL penicillin, 100 μg/mL streptomycin, 10 μg/mL gentamycin and IL-2 (40 U/mL, R&D Systems, Minneapolis, Minn.). Fifty microtiters of cells were then distributed to the inner 60 wells of a 96 well round bottom microtiter culture plate, and serially diluted compounds K and L were added to the Microtiter plate. All samples were assayed in triplicate with a replicate plate without virus for the determination of compound toxicity. The final volume per well was 200 μL. The assay was incubated for 6 days in a humidified atmosphere at 37° C., 5% CO₂, the plates analyzed for cell viability by MTS dye reduction or incorporation of [³H] metabolite incorporation.

[³H] Metabolite Incorporation

[³H]Uridine, [³H] thymidine or [³H]leucine incorporation were measured in the following manner. Twenty-four hours prior to termination of the assay 0.001 μCi of the appropriate labeled metabolite was added per well. Incorporation was then determined on a Wallac Microbeta counter following lysis of the cells with H₂O and capture on glass fiber filters using a Skatron harvester.

MTS Staining for Cell Viability

At assay termination the assay plates were stained with the soluble tetrazolium-based dye MTS (CellTiter® Reagent, Promega) to determined cell viability and quantify compound toxicity. MTS is metabolized by the mitochondria enzymes of metabolically active cells to a soluble formazan product, allowing the rapid quantitative analysis cell viability and compound cytotoxicity. This reagent is a single stable solution that does not require preparation before use. At termination of the assay 20 μL of MTS reagent was added per well. The wells were incubated for 4 h at 37° C., 5% CO₂. Adhesive plate scalers were used in place of the lids, the sealed plate was inverted several times to mix the soluble formazan product and the plate was read spectrophotometrically at 490 nm with a 650 nm reference wavelength on a Molecular Devices Vmax plate reader.

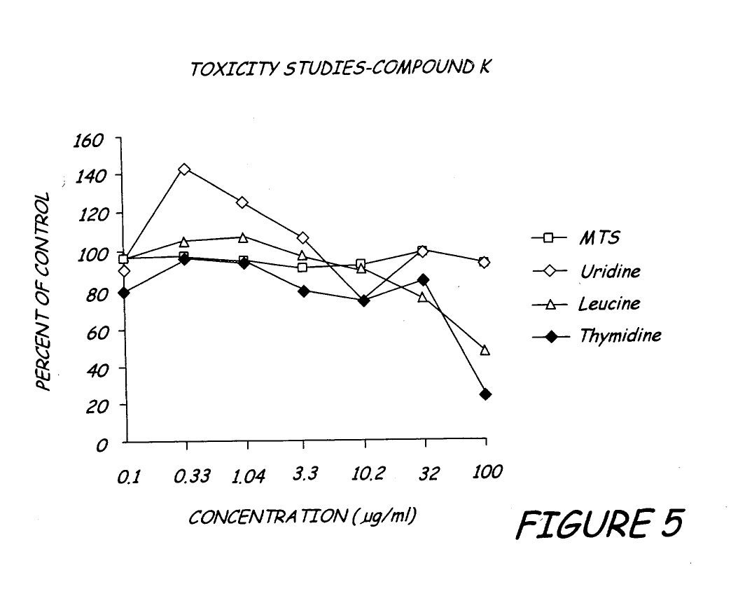

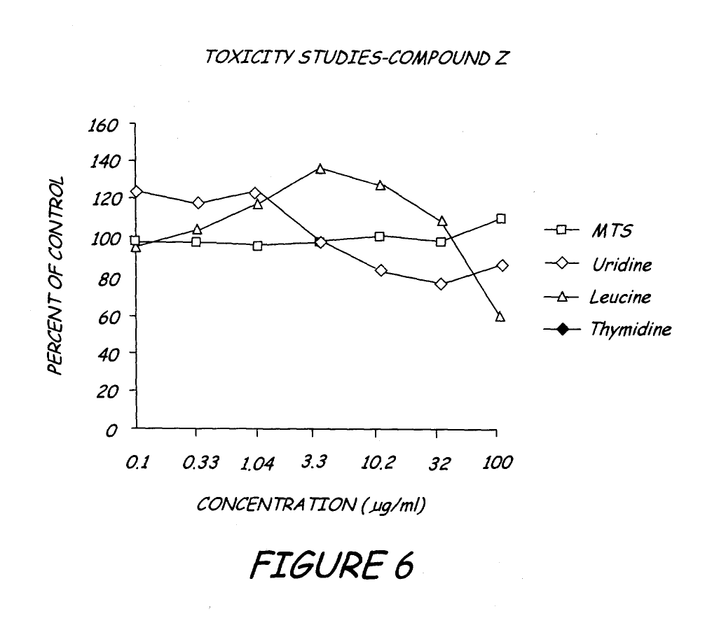

The results are shown in FIGS. 5 and 6. Both compounds K and Z were tested at a high test concentration of 100 μg/ml. In previous studies compound K and Z were non-toxic to PBMCs at concentrations above 400 μg/ml and exhibited IC₅₀'s below 50 μg/ml. Thus the concentrations were selected to cover this range. For both compounds (3H) thymidine uptake as a measurement of DNA synthesis showed a close correlation to MTS dye reduction. The MTS dye reduction was less variable in outcome over the concentration range, possibly due to the experimental errors introduced using the cell harvester. For compound K (FIG. 5) both protein (leucine incorporation) and RNA (uridine incorporation) production start to drop above 32 μg/ml. The previously identified TC₅₀ in PBMCs by MTS was 499 μg/ml. This suggests that the drops seen in protein and RNA incorporation may be preceding the cytotoxic effects of compound K are reflecting disruption of general cell transcription. Compound Z (FIG. 6) showed no effect on DNA or RNA metabolism at 100 μg/ml, however protein incorporation was beginning to drop at 100 μg/ml (40% inhibition.) Thus determination of cell viability by the MTS method is valid for compound K and Z within the concentration range where antiviral activity is observed. Additionally, the metabolic effects of compound K and Z in the concentration range associated with antiviral activity are minimal, and are believed to either reflect independence of the compounds antiviral action from metabolic functions or selectivity for virus targets over cell targets.

Assessing Compound Z for Antiviral Activity in the U1 and ACH-2 Assay U1 and ACH-2 Assays

U1 and ACH-2 cells were obtained from the AIDS Research and Reference Reagent Program and maintained under standard culture conditions in RPMI 1640 supplemented with 10% fetal bovine serum (heat inactivated), supplemented with 2 mM L-glutamate, 100U/ml penicillin and 100 μg/ml, streptomycin. U1 cells were derived from the histocytic leukemia cell line U937, and contain a single integrated copy (HIV IIIB) or a cytokine and/or phorbol inducible provirus. ACH-2 cells are derived from the A3.01 T lymphoblastic cell line, and contain 2 copies of the HIV IIIB provirus, however only one is cytokine and/or phorbol inducible. Cultures were maintained in such a way as to ensure exponential growth of the populations. At the time of the assay, cells were collected by centrifugation and counted by hemacytometer. If cell viability by Trypan Blue dye exclusion was less than 70% the assay was terminated. The cells were adjusted to 5×10⁴ cells/mL and 100 μL, placed in 96 well plates with 100 μL media containing a final concentration of 5 μg/mL TNFα and the test compound. Cultures were incubated for 3 days and supernatants harvested. Compound toxicity was determined by MTS dye reduction. Virus expression was measured by supernatant reverse transcriptasc activity.

Reverse Transcriptase Assay

Reverse transcriptase activity was measured in cell-free supernatants. Tritiated thymidine triphosphate (NEN) (TTP) was resuspended in distilled H₂O at 5 Ci/mL. Poly rA and oligo dT were prepared as a stock solution which was kept at −20° C. The RT reaction buffer was prepared fresh on a daily basis and consists of 125 μL 1.0 M EGTA, 125 μL dH₂O, 110 μL 10% SDS, 50 μL 1.0 M Tris (pH 7.4), 50 μL, 1.0 M DTT, and 40 μL 1.0 M MgCl₂. These three solutions were mixed together in a ratio of 2 parts TTP, 1 part poly rA:oligo dT, and 1 part reaction buffer. Ten microliters of this reaction mixture was placed in a round bottom microtiter plate and 15 μL of virus containing supernatant was added and mixed. The plate was incubated at 37° C. in a water bath with a solid support to prevent submersion of the plate and incubated for 60 minutes. Following reaction, the reaction volume was spotted onto pieces of DE81 paper, washed 5 times for 5 minutes each in a 5% sodium phosphate buffer, 2 times for 1 minute each in distilled water, 2 times for 1 minute each in 70% ethanol, and then dried. Opti-Fluor O was added to each sample and incorporated radioactivity was quantitated utilizing a Wallac 1450 Microbetaplus liquid scintillation counter.

### Results

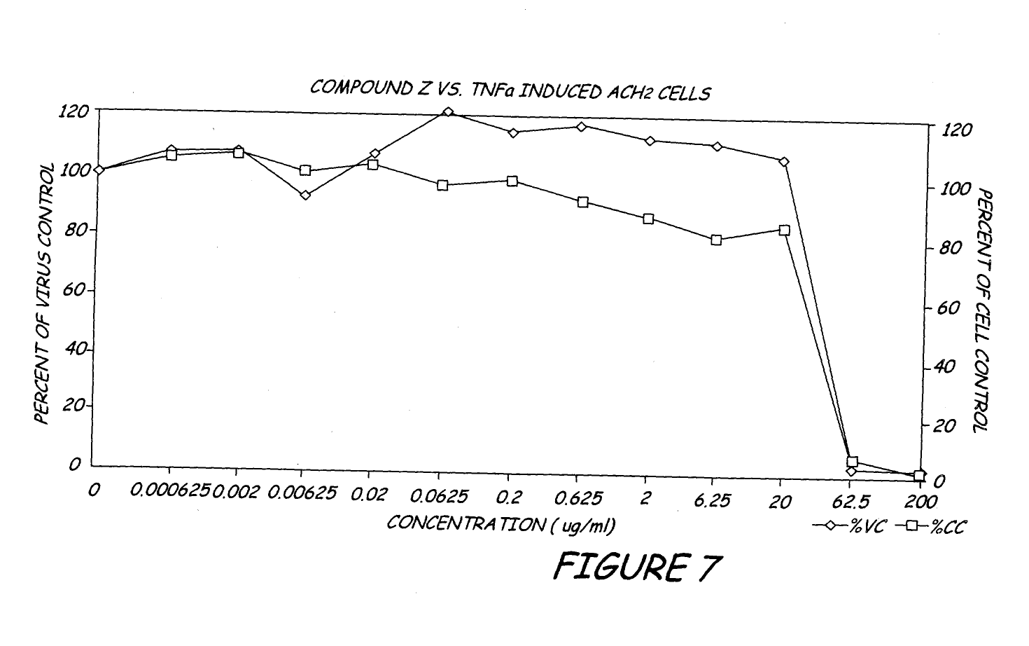

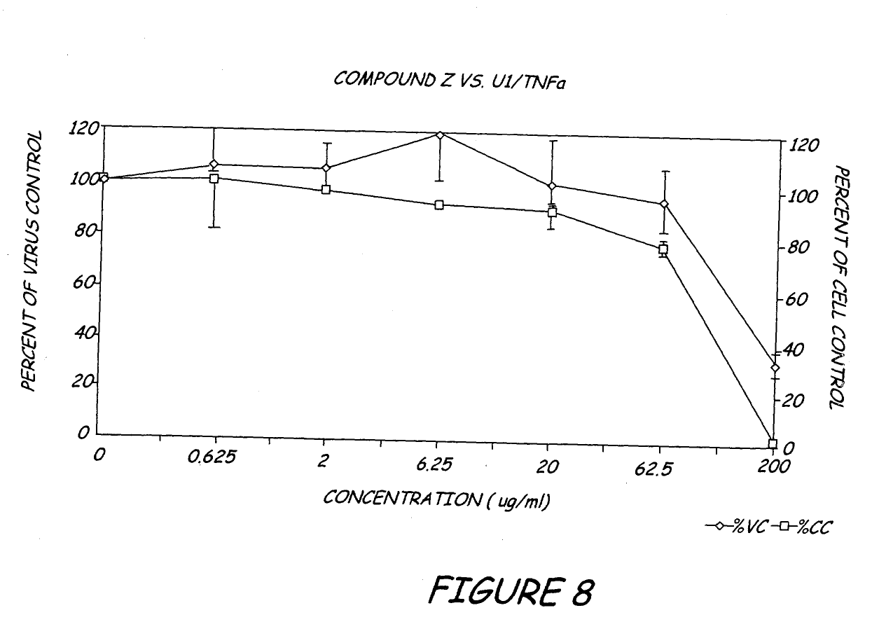

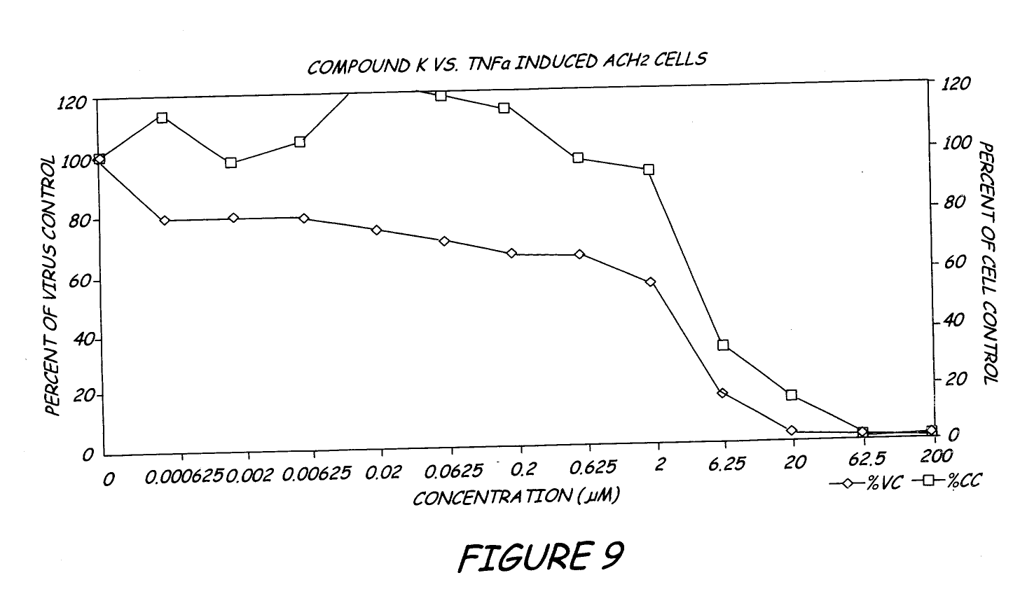

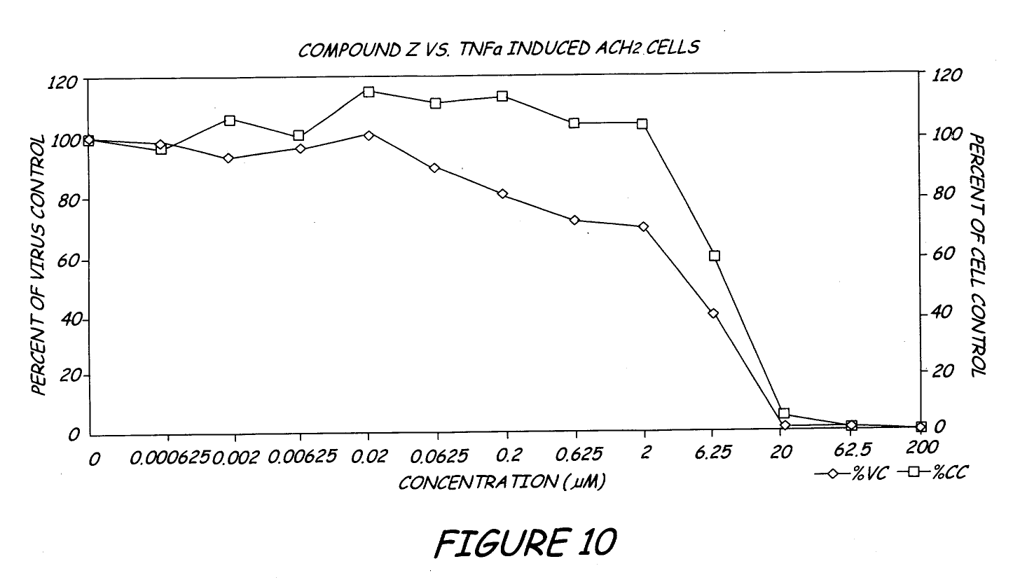

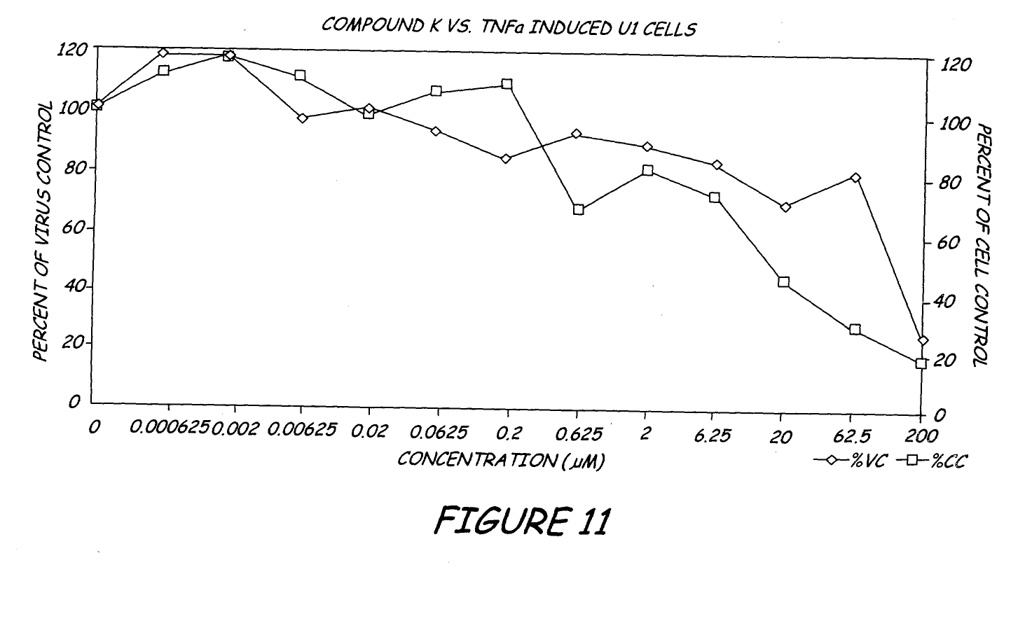

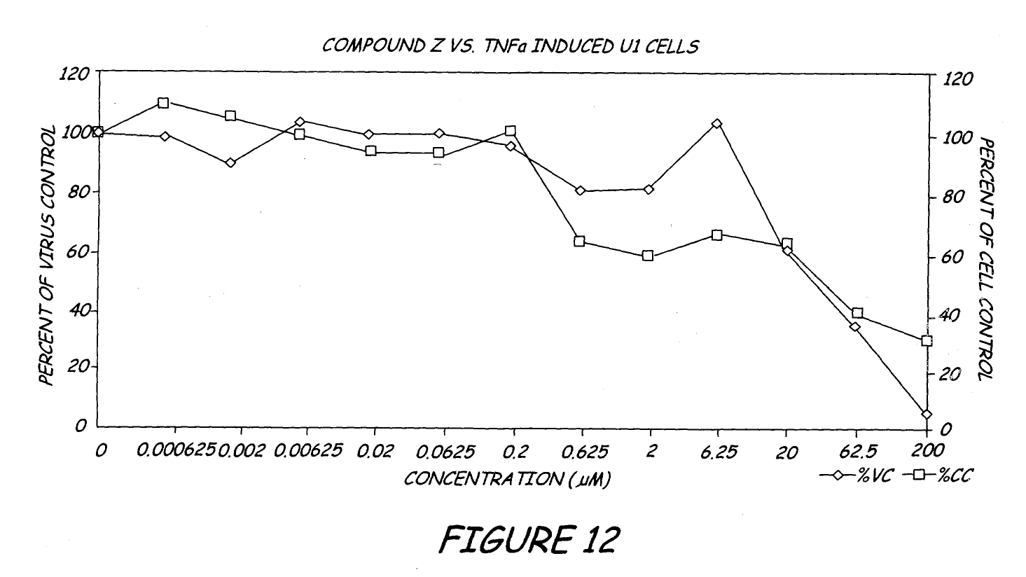

The results of multiple tests are summarized in Table 1 and the raw data are included in Tables 2 through 9 and FIGS. 7 through 12. Compound Z was initially tested against both U1 and ACH-2 cells (experiment 1). In both cases the cytotoxicity paralleled antiviral efficacy. Compounds K and Z were run in parallel (experiment 2). Compound K inhibited virus replication in U1 cells with an IC₅₀ of 17.7 μg/ml and a TI of 7.9.

Re-examination of the previous experiments and comparison with the current data suggests that antiviral efficacy for compound K in both U1 and ACH-2 cells maybe in close balance with the cytotoxic effects of the compound. This is a phenomena encountered previously and can be thought of as resulting from micro-factors in the tissue culture system. Thus intra-experiment variations result in a pendulum effect due to the close relationship of cyotoxicity and efficacy. This does not invalidate the observations of activity in this system, but rather makes it more difficult to use the U1 and ACH-2 system for mechanistic studies.

TABLE 1
UI and ACH-2 Assay Results

| | | Compound K | | | Compound Z | | |
|---|---|---|---|---|---|---|---|
| Experiment | Cell type | 1C₅₀ | TC₅₀ | TI | I₅₀ | TC₅₀ | T1 |
| 1 | U1 | | 1 | | 164 | 114 | — |
| | ACH- | | | | 43.5 | 39 | — |
| 2 | U1 | 17.7 | 140.1 | 7.9 | 44 | 39 | — |
| | ACH-2 | 2.5 | 5 | 2 | 8.9 | 4.8 | — |

Time of Addition Assay for Compound Z

Time of addition assays were undertaken for an initial determination of compound mechanism of action.

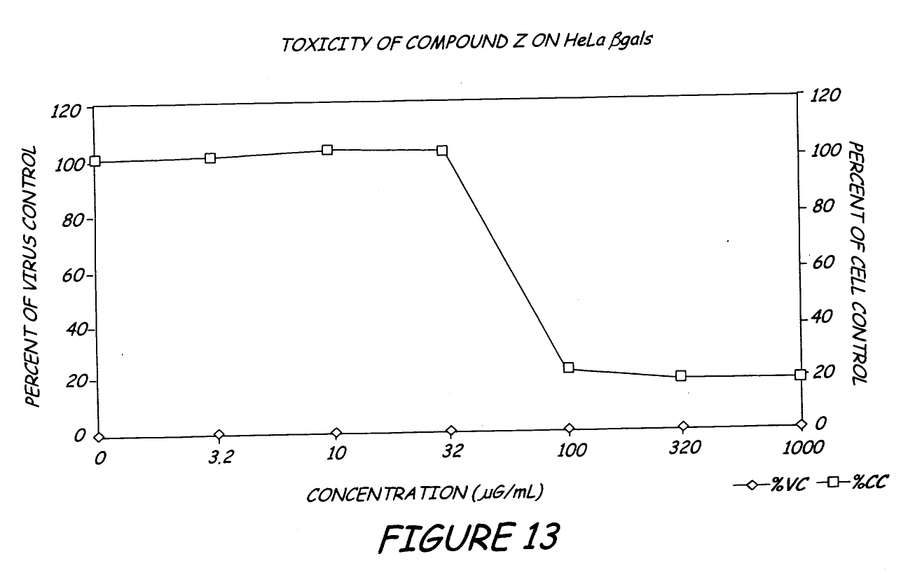

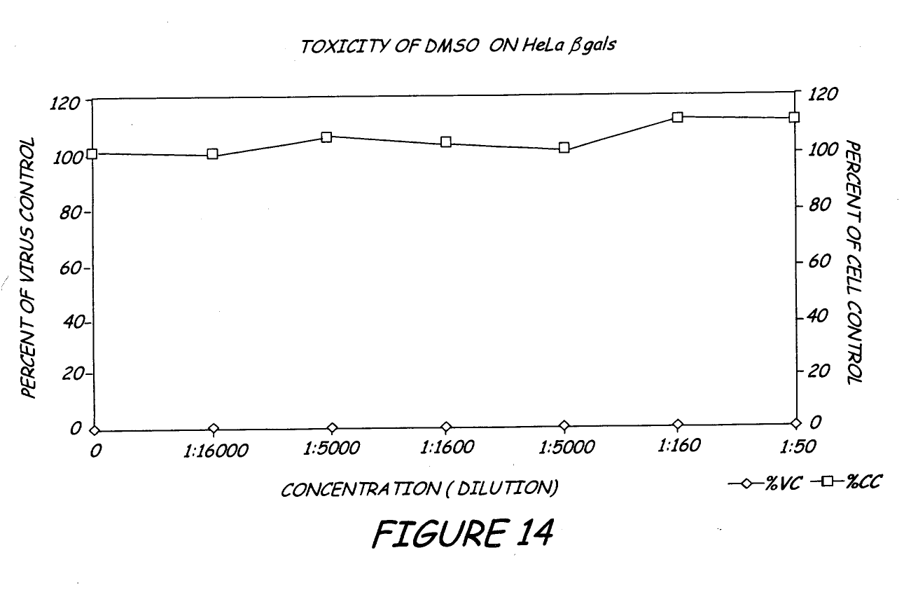

An initial test experiment was performed to determine if compound Z was cytotoxic for HeLa CD4 LTR β-gal cells. This cell line represents one of 3 standard models for performing time of addition assays. As seen in Table 9 the TC₅₀ in this model was 75 μg/ml. Compound Z tested for efficacy in HeLa CD4 LTR β-gal attachment assay, and was found to be inactive with a TC₅₀ greater than 100 μg/ml Tables 10 and 11 and FIGS. 13 and 14.

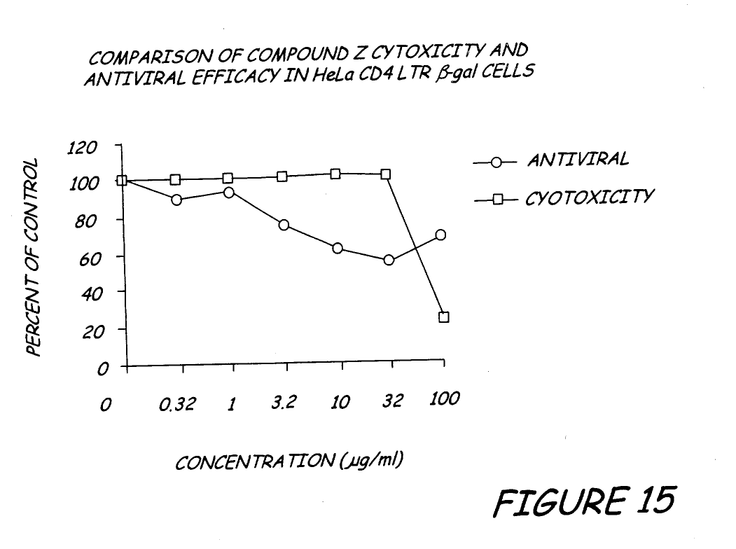

FIG. 15 shows a composite graph of efficacy and cytotoxicity, that demonstrates compound Z's toxicity could be potentially separated from antiviral activity, making this a model for the time of addition assay. In addition this experiment also demonstrates that compound Z does not appear to inhibit a target early in HIV replication. The design of the HeLa CD4 LTR β-gal assay is such that compounds are added during the attachment phase of virus replication and removed immediately after (1 h post infection). The lack of significant antiviral activity demonstrates that the antiviral target may not be a virus attachment inhibitor. The dip in the antiviral efficacy curve also suggests that there is a potential target for activity in the HeLa CD4 LTR β-gal cells that the design of the experiment (compound removal at 1 h post infection) is being not allowing to maximize.

TABLE 2
COMPOUND Z VS. LATENTLY INFECTED ACH2 CELLS

| | | | | | | | | | | | | | |
|---|---|---|---|---|---|---|---|---|---|---|---|---|---|
| CONC (μG/Ml) | 200 | 62.5 | 20 | 6.25 | 2 | 0.625 | 0.2 | 0.0625 | 0.002 | 0.00625 | 0.002 | 0.00063 | 0 |

RT ACTIVITY (cpm)

| | | | | | | | | | | | | | |
|---|---|---|---|---|---|---|---|---|---|---|---|---|---|
| SAMPLE 1 | 2251 | 3272 | 71330 | 71476 | 70152 | 75627 | 78196 | 85463 | 76329 | 68819 | 77842 | 84914 | 63234 |
| SAMPLE 2 | 1053 | 1962 | 75077 | 76104 | 81623 | 84265 | 76355 | 79225 | 69043 | 49680 | 66035 | 61574 | 67688 |
| SAMPLE 3 | 1157 | 1325 | 69781 | 78378 | 76599 | 78372 | 78012 | 82364 | 70571 | 68515 | 71652 | 69173 | 69488 |
| MEAN | 1487 | 2186 | 72063 | 75319 | 76125 | 79421 | 77521 | 82351 | 71981 | 62338 | 71843 | 71887 | 66803 |
| % VC | 2.23 | 3.27 | 107.9 | 112.7 | 114.0 | 118.9 | 116.0 | 123.3 | 107.8 | 93.3 | 107.5 | 107.6 | 100.0 |

TOXICITY VALUES (MTS - O. D. @ 4901650 nm)

| | | | | | | | | | | | | | |
|---|---|---|---|---|---|---|---|---|---|---|---|---|---|
| SAMPLE 1 | 0.015 | 0.063 | 0.813 | 0.83 | 0.904 | 0.936 | 1.059 | 0.992 | 1.018 | 1.023 | 1.089 | 1.048 | 0.991 |
| SAMPLE 2 | 0.007 | 0.062 | 0.849 | 0.815 | 0.89 | 0.928 | 1.021 | 1.005 | 1.082 | 1.015 | 1.072 | 1.057 | 0.997 |
| SAMPLE 3 | 0.016 | 0.065 | 0.906 | 0.821 | 0.862 | 0.952 | 0.926 | 0.955 | 1.048 | 1.02 | 1.083 | 1.079 | 1.028 |
| MEAN | 0.013 | 0.063 | 0.856 | 0.822 | 0.885 | 0.939 | 1.002 | 0.984 | 1.049 | 1.019 | 1.081 | 1.061 | 1.005 |
| % CC | 1.3 | 6.3 | 85.1 | 81.8 | 88.1 | 93.4 | 99.7 | 97.9 | 104.4 | 101.4 | 107.6 | 105.6 | 100.0 |

IC50 (μG/Ml) = 43.51
ITC50 (μG/Ml) = 38.94
TI > NA

TABLE 3
Compound Z Versus U1/TNFα Latent Infection
RT VALUES (cpm)

| CONC (Ngfml) | O | 0.625 | 2 | 6.25 | 20 | 62.5 | 200 |
|---|---|---|---|---|---|---|---|
| SAMPLE I | 2607.4 | 2944.4 | 2651.4 | 2915.4 | 2723.4 | 2175.4 | 737.4 |
| SAMPLE 2 | 2191.4 | 2767.4 | 2595.4 | 3164.4 | 2463.4 | 2563.4 | 745.4 |
| SAMPLE 3′ | 2271.4 | 1802.4 | 2231.4 | 2359.4 | 1970.4 | 2022.4 | 925.4 |
| MEAN | 2356.7 | 2504.7 | 2492.7 | 2813.0 | 2385.7 | 2253.7 | 802.7 |
| X VC | 100.0 | 106.3 | 105.8 | 119.4 | 101.2 | 95.6 | 34.1 |
| STD DEV | 9.4 | 26.1 | 9.7 | 17.5 | 16.2 | 11.8 | 4.5 |

TOXICITY VALUES (MTS - O. D. @ 490/650 nm)

| CONC (Aphnl) | 0 | 0.625 | 2 | 6.25 | 20 | 62.5 | 200 |
|---|---|---|---|---|---|---|---|
| SAMPLE I | 1.789 | 1.791 | 1.724 | 1.668 | 1.637 | 1.402 | 0.060 |
| SAMPLE 2 | 1.770 | 1.740 | 1.757 | 1.657 | 1.644 | 1.324 | 0.056 |
| SAMPLE 3 | 1.757 | 1.841 | 1.715 | 1.623 | 1.594 | 1.412 | 0.073 |
| MEAN | 1.772 | 1.791 | 1.732 | 1.649 | 1.625 | 1.379 | 0.063 |
| % CC | 100.0 | 101.1 | 97.7 | 93.1 | 91.7 | 77.8 | 3.6 |
| STD DEV | 0.9 | 2.8 | 1.2 | 1.3 | 1.5 | 2.7 | 0.5 |

IC50 (ug/ml) = ..164.40
IITC5O (ug/ml) = 114.0320334
T1 = NA

TABLE 4
Evaluation Versus Latently Infected Cells

| | | | Therapeutic |
|---|---|---|---|
| Compound | ACH2 EC₅₀ | ACH2 TC₅₀ | Index |
| K (μM) | 2.5 | 5.0 | — |
| Z (μg/ml) | 8.9 | 4.8 | — |
| | | | Therapeutic |
| Compound | U1 EC₅₀ | U1 TC₅₀ | Index |
| K (μM) | 17.7 | 140.1 | 7.9 |
| Z (μg/ml) | 44.0 | 38.8 | — |

TABLE 5
COMPOUND K VS. LATENTLY INFECTED ACH2 CELLS
RT ACTIVITY (cpm)

| CONC 61M) | 200 | 62.5 | 20 | 6.25 | 2 | 0.625 | 0.2 | 0.0625 | 0.02 | 0.00625 | 0.002 | 0.00063 | 0 |
|---|---|---|---|---|---|---|---|---|---|---|---|---|---|
| SAMPLE I | 132 | 136 | 7159 | 4935 | 19927 | 24384 | 22992 | 23579 | 25143 | 19750 | 18606 | 21198 | 18961 |
| SAMPLE 2 | 80 | 108 | 1238 | 7199 | 11798 | 20127 | 22189 | 24006 | 26273 | 21650 | 19486 | 24484 | 20921 |
| SAMPLE 3 | 112 | 96 | 637 | 7163 | 17518 | 13055 | 22598 | 22623 | 22630 | 20617 | 19646 | 21727 | 19044 |
| MEAN | 108 | 113 | 3011 | 6432 | 18381 | 19189 | 22593 | 23403 | 24682 | 20672 | 19246 | 22470 | 19642 |
| 7h VC | 0.55 | 0.58 | 15.3 | 32.7 | 93.6 | 97.7 | 115.0 | 119.1 | 125.7 | 105.2 | 98.0 | 114.4 | 100.0 |

TOXICITY VALUES (MTS - O. D. @ 490/650 nm)

| CONC (PM) | 200 | 62.5 | 20 | 6.25 | 2 | 0.625 | 0.2 | 0.0625 | 0.02 | 0.00625 | 0.002 | 0.00063 | 0 |
|---|---|---|---|---|---|---|---|---|---|---|---|---|---|
| SAMPLE 1 | 0.054 | 0 | 0.006 | 0.143 | 0.67 | 0.799 | 0.7 | 0.879 | 0.974 | 1.014 | 1.018 | 0.982 | 1.146 |
| SAMPLE 2 | 0.031 | 0.034 | 0.051 | 0.234 | 0.698 | 0.844 | 0.887 | 0.921 | 0.961 | 1.016 | 1.02 | 1.004 | 1.387 |
| SAMPLE 3 | 0 | 0.026 | 0.063 | 0.246 | 0.713 | 0.835 | 0.904 | 0.873 | 0.893 | 0.97 | 0.974 | 0.984 | 1.267 |
| MEAN | 0.028 | 0.020 | 0.040 | 0.208 | 0.694 | 0.826 | 0.830 | 0.891 | 0.943 | 1.000 | 1.004 | 0.990 | 1.267 |
| Y. CC | 2.2 | 1.6 | 3.2 | 16.4 | 54.8 | 65.2 | 65.6 | 70.3 | 74.4 | 78.9 | 79.3 | 78.2 | 100.0 |

IC50 (4M) = 5.0
TC50 (uM) = 2.53
TI > NA

TABLE 6
COMPOUND Z VS. LATENTLY INFECTED ACH2 CELLS
RT ACTIVITY (rpm)

| CONC (Nfllml) | 200 | 62.5 | 20 | 6.25 | 2 | 0.625 | 0.2 | 0.0625 | 0.02 | 0.00625 | 0.002 | 0.00063 | 0 |
|---|---|---|---|---|---|---|---|---|---|---|---|---|---|
| SAMPLE I | 96 | 172 | 396 | 9104 | 19441 | 19417 | 23953 | 22369 | 22622 | 18489 | 19365 | 18746 | 19156 |
| SAMPLE 2 | 96 | 156 | 1293 | 13315 | 18160 | 21148 | 21338 | 21353 | 22149 | 20581 | 20032 | 18421 | 19971 |
| SAMPLE 3 | 48 | 152 | 1257 | 12769 | 22852 | 19758 | 20659 | 20875 | 22374 | 19164 | 22152 | 18614 | 18742 |
| MEAN | 80 | 160 | 982 | 11729 | 20151 | 20108 | 21983 | 21532 | 22382 | 19411 | 20516 | 18594 | 19290 |
| % VC | 0.41 | 0.83 | 5.1 | 60.8 | 104.5 | 104.2 | 114.0 | 111.6 | 116.0 | 100.6 | 106.4 | 96.4 | 100.0 |

TOXICITY VALUES (MTS - O, p. @ 490/650 nm)

| CONC (,igrmi) | 200 | 62.5 | 20 | 6.25 | 2 | 0.625 | 0.2 | 0.0625 | 0.02 | 0.00626 | 0.002 | 0.00063 | 0 |
|---|---|---|---|---|---|---|---|---|---|---|---|---|---|
| SAMPLE 1 | 0 | 0 | 0 | 0.344 | 0.666 | 0.707 | 0.819 | 0.885 | 0.931 | 0.939 | 0.96 | 1.02 | 0.993 |
| SAMPLE 2 | 0 | 0 | 0 | 0.409 | 0.67 | 0.714 | 0.828 | 0.909 | 1.062 | 0.957 | 0.883 | 0.924 | 1.111 |
| SAMPLE 3 | 0 | 0 | 0 | 0.445 | 0.753 | 0.734 | 0.773 | 0.888 | 1.025 | 0.986 | 0.943 | 0.982 | 0.882 |
| MEAN | 0.000 | 0.000 | 0.000 | 0.399 | 0.696 | 0.718 | 0.807 | 0.894 | 1.006 | 0.961 | 0.929 | 0.975 | 0.995 |
| 9′. CC | 0.0 | 0.0 | 0.0 | 40.1 | 70.0 | 72.2 | 81.0 | 89.8 | 101.1 | 96.5 | 93.3 | 98.0 | 100.0 |

IC50 (wg/ml) = 8.92
TC50 (vg/mol) = 4.84
Tl > NA

TABLE 7
COMPOUND K VS. LATENTLY INFECTED U1 CELLS
RT ACTIVITY (cpm)

| | | | | | | | | | | | | | |
|---|---|---|---|---|---|---|---|---|---|---|---|---|---|
| CONC (μM) | 200 | 62.5 | 20 | 6.25 | 2 | 0.625 | 0.2 | 0.0625 | 0.02 | 0.00625 | 0.002 | 0.00063 | 0 |

TOXICITY VALUES (MTS - O. D. @ 490/650 nm)

| CONC (μM) | 200 | 62.5 | 20 | 6.25 | 2 | 0.625 | 0.2 | 0.0625 | 0.2 | 0.00625 | 0.002 | 0.00063 | 0 |
|---|---|---|---|---|---|---|---|---|---|---|---|---|---|
| SAMPLE 1 | 0.351 | 1.054 | 0.912 | 1.017 | 1.239 | 1.143 | 1.112 | 1.274 | 1.362 | 1.319 | 1.431 | 1.493 | 1.335 |
| SAMPLE 2 | 0.353 | 1.078 | 0.926 | 1.128 | 1.078 | 1.219 | 1.152 | 1.208 | 1.368 | 1.228 | 1.593 | 1.485 | 1.228 |
| SAMPLE 3 | 0.32 | 1.019 | 0.902 | 1.105 | 1.157 | 1.279 | 1.045 | 1.184 | 1.23 | 1.266 | 1.634 | 1.661 | 1.337 |
| MEAN | 0.341 | 1.050 | 0.913 | 1.083 | 1.158 | 1.214 | 1.103 | 1.222 | 1.320 | 1.271 | 1.553 | 1.546 | 1.300 |
| % CC | 26.3 | 80.8 | 70.3 | 83.3 | 89.1 | 93.4 | 84.8 | 94.0 | 101.5 | 97.8 | 119.4 | 118.9 | 100.0 |
| SAMPLE 1 | 476 | 749 | 881 | 1966 | 2106 | 1890 | 2777 | 2301 | 2671 | 3127 | 4225 | 3239 | 2898 |
| SAMPLE 2 | 372 | 592 | 1433 | 1345 | 2847 | 1621 | 3556 | 3087 | 3102 | 3119 | 2927 | 3279 | 2701 |
| SAMPLE 3 | 560 | 997 | 1333 | 2574 | 1621 | 1966 | 2514 | 3227 | 2146 | 2722 | 2370 | 2538 | 2445 |
| MEAN | 469 | 779 | 1216 | 1962 | 2191 | 1826 | 2949 | 2872 | 2640 | 2989 | 3174 | 3019 | 2681 |
| % VC | 17.50 | 29.07 | 45.3 | 73.2 | 81.7 | 68.1 | 110.0 | 107.1 | 98.4 | 111.5 | 118.4 | 112.6 | 100.0 |

IC50 (fLM) = 17.70
TC50 (pM) = 140.14
T1 = 7.92

TABLE 8
COMPOUND Z VS. LATENTLY INFECTED U1 CELLS
RT ACTIVITY (cpm)

| CONC (POW) | 200 | 62.5 | 20 | 6.25 | 2 | 0.625 | 0.2 | 0.0625 | 0.02 | 0.00625 | 0.002 | 0.00063 | 0 |
|---|---|---|---|---|---|---|---|---|---|---|---|---|---|
| SAMPLE I | 658 | 1073 | 2082 | 2518 | 1814 | 1746 | 3235 | 2983 | 2975 | 3824 | 3518 | 3584 | 3540 |
| SAMPLE 2 | 1413 | 1533 | 2923 | 2719 | 1946 | 1445 | 2603 | 2433 | 2899 | 2763 | 3154 | 3103 | 2142 |
| SAMPLE 3 | 778 | 1069 | 837 | 941 | 1753 | 2761 | 3508 | 3256 | 2831 | 2643 | 3063 | 3480 | 3556 |
| MEAN | 950 | 1225 | 1947 | 2059 | 1838 | 1984 | 3115 | 2891 | 2902 | 3077 | 3245 | 3.389 | 3079 |
| % VC | 30.84 | 39.78 | 63.2 | 66.9 | 59.7 | 64.4 | 101.2 | 93.9 | 94.2 | 99.9 | 105.4 | 110.1 | 100.0 |

TOXICITY VALUES (NITS - O. D. @ 490/650 nm)

| conic (,ymq | 200 | 6.25 | 20 | 6.25 | 2 | 0.625 | 0.2 | 0.0625 | 0.02 | 0.00625 | 0.002 | 0.00063 | 0 |
|---|---|---|---|---|---|---|---|---|---|---|---|---|---|
| SAMPLE 1 | 0.143 | 0.371 | 0.614 | 1.116 | 0.92 | 0.033 | 1.022 | 1.04 | 1.036 | 1.109 | 0.918 | 1.039 | 1.023 |
| SAMPLE 2 | 0.084 | 0.345 | 0.775 | 1.124 | 0.89 | 0.889 | 1.049 | 1.104 | 1.091 | 1.122 | 0.865 | 1.063 | 0.983 |
| SAMPLE 3 | 0 | 0.411 | 0.58 | 1.114 | 0.817 | 0.882 | 1.023 | 1.071 | 1.064 | 1.077 | 1.107 | 1.064 | 1.187 |
| MEAN | 0.076 | 0.376 | 0.658 | 1.118 | 0.876 | 0.868 | 1.031 | 1.072 | 1.064 | 1.103 | 0.963 | 1.055 | 1.064 |
| % CC | 7.1 | 35.3 | 61.7 | 105.0 | 82.3 | 81.6 | 96.9 | 100.7 | 99.9 | 103.6 | 90.5 | 99.2 | 100.0 |

IC50 (uglml) = 43.99
TC50 (uglml) = 38.80
TI = NA

TABLE 9

| COMPOUND | TC₅₀ |
|---|---|
| Compound Z | 76.5/ml |
| DMSO | 1:50 dilution |

Visual Observation: Toxicity was visually seen in the first three dilutions, The monolayer was not apparent in the first two dilutions and in the 3^{rd} dilution it was about 50% confluent. The monolayer was about 90% confluent by the 4^{th} dilution

TABLE 10
Toxicity of Compound Z on HeLa β-gals

| | | | | | | | |
|---|---|---|---|---|---|---|---|
| CONC (μG/mL) | 0 | 3.2 | 10 | 32 | 100 | 320 | 1000 |

RLU (Relative Light Units)

| | | | | | | | |
|---|---|---|---|---|---|---|---|
| SAMPLE 1 | #DIV/10 | #DIV/01 | #DIV/01 | #DIV/01 | #DIV/01 | #DIV/01 | #DIV/01 |
| SAMPLE 2 | #DIV/10 | #DIV/01 | #DIV/01 | #DIV/01 | #DIV/01 | #DIV/01 | 4DIV/01 |
| SAMPLE 3 | #DIV/01 | #DIV/01 | #DIV/01 | #DIV/01 | #DIV/01 | #DIV/01 | #DIV/01 |
| MEAN | #DIV/01 | #DIV/01 | #DIV/01 | #DIV/01 | #DIV/01 | #DIV/01 | #DIV/01 |
| % VC | #DN/10 | #DIV/01 | #DIV/01 | #DIV/01 | #DIV/01 | #DIV/01 | #DIV/01 |

TOXICITY VALUES (Cell Titer - O. D. @ 490/650 nm)

| | | | | | | | |
|---|---|---|---|---|---|---|---|
| SAMPLE 1 | 1.158 | 1.396 | 1.229 | 1.142 | 0.282 | 0.210 | 0.228 |
| SAMPLE 2 | 1.162 | 1.039 | 1.167 | 1.155 | 0.237 | 0.207 | 0.205 |
| SAMPLE 3 | 1.111 | 1.033 | 1.131 | 1.193 | 0.256 | 0.217 | 0.189 |
| MEAN | 1.144 | 1.156 | 1.176 | 1.164 | 0.259 | 0.212 | 0.208 |
| % CC | 100.0 | 101.1 | 102.8 | 101.7 | 22.6 | 18.5 | 18.2 |

IC₅₀ (μG/mL) = #DIV/01 I
TC50 (NGImL) = 76.5
TI = NIA

TABLE 11
Toxicity of DMSO on HeLa β-gals
RLU (Relative Light Units)

| CONC (dilution) | 0 | 1:10000 | 1:5000 | 1:1600 | 1:500 | 1:160 | 1:60 |
|---|---|---|---|---|---|---|---|
| SAMPLE 1 | #DIV/01 | #DIV/01 | #DIV/01 | #DIV/01 | #fDIV/01′ | #DIV/01 | #DIV/01 |
| SAMPLE 2 | #DIV/01 | #DIV/01 | #DIV/01 | #DIV/01 | #DIV/01 | #DIV/01 | #DIV/01 |
| SAMPLE 3 | #DIV/01 | #DIV/01 | #DIV/01 | #DIV/01 | #DIV/01 | #DIV/01 | #DIV/01 |
| MEAN | #DIV/011 | #DIV/01 | #DIV/01 | #DIV/01 | #DIV/01 | #DIVI01 | #DIV/01 |
| % VC | #DIV/01I | #DIV/01 | #DIV/OI | #DIV/01 | #DIV/01 | #DIV/01 | #DIV/01 |

TOXICITY VALUES (Cell Titer- O. D. @ 490/650 nm)

| CONC (dilution) | 0 | 1:16000 | 1:5000 | 1:1600 | 1.500 | 11110 | 1:50 |
|---|---|---|---|---|---|---|---|
| SAMPLE 1 | 0.910 | 1.037 | 1.096 | 1.100 | 1.043 | 1.349 | 1.210 |
| SAMPLE 2 | 1.165 | 0.999 | 1.151 | 1.193 | 1.061 | 1.062 | 1.292 |
| SAMPLE 3 | 1.078 | 1.090 | 1.091 | 0.961 | 1.081 | 1.090 | 0.999 |
| MEAN | 1.051 | 1.042 | 1.113 | 1.085 | 1.062 | 1.167 | 1.167 |
| % CC | 100.0 | 99.1 | 105.9 | 103.2 | 101.0 | 111.0 | 111.0 |

C₅₀ (dilution) #DIV/01
ITC₅₀ (dilution) = >1:50
TI = N/A

The following observations can be drawn from the following experimentals:

1. Interference of the Iodiscorbate Compounds with the CellTiter® Reagent

The measurement of cell viability and survival of PBMCs by MTT paralleled ³H thymidine, uridine and leucine incorporation for compounds K and Z up to 100 μg/ml. At 100 μg/ml compound K thymidine incorporation (DNA synthesis) decreased, however compound Z showed no significant effect on cell division. The cells are viable and antiviral activity is a reflection of the inhibition of virus replication not cell toxicity.

2. Activity of Compound Z in Latently Infected U1 and ACH-2 Cells

Compound Z was found to be consistently inactive in the U1 and ACH-2 assay due to cytotoxicity of the compound. However, this may indicate a transcriptional or post-integration target since these cells are often sensitive agents with activity for transformed cells. Thus, the U1 and ACH-2 assays suggest a post-integrative antiviral target for compound K.

3. Testing of Compound K and Z in Chronically Infected Cells

In these studies PBMCs are infected with HIV RoJo (a clinical isolate) and a dynamic population of infected PBMCs established over a period of 2 to 3 weeks. This population of infected PBMCs was established and maintained by the addition of fresh PBMCs in a 1:1 ratio every 7 days, thus overcoming the lost of PBMCs to normal cell death processes. Thus a population of cells was established that consisted of uninfected cells, short lived infected cells and long lived infected cells. In experiments where AZT was added to prevent re-infection of the population, we typically saw a 50% reduction in infection following a 7 day treatment. This means that 50% of the virus production from the infected PI 3MC population was due re-infection. Extension of the AZT to 14 or 21 days resulted in the reduction of virus expression by 90 to 95%. Thus there was a population of infected PBMCs present (5 to 10′/a of the total) which survived for extended periods of time and thus met the definition of being chronically infected. It was this model which was used to assess the effects of compound K acid Z on chronically infected cells.

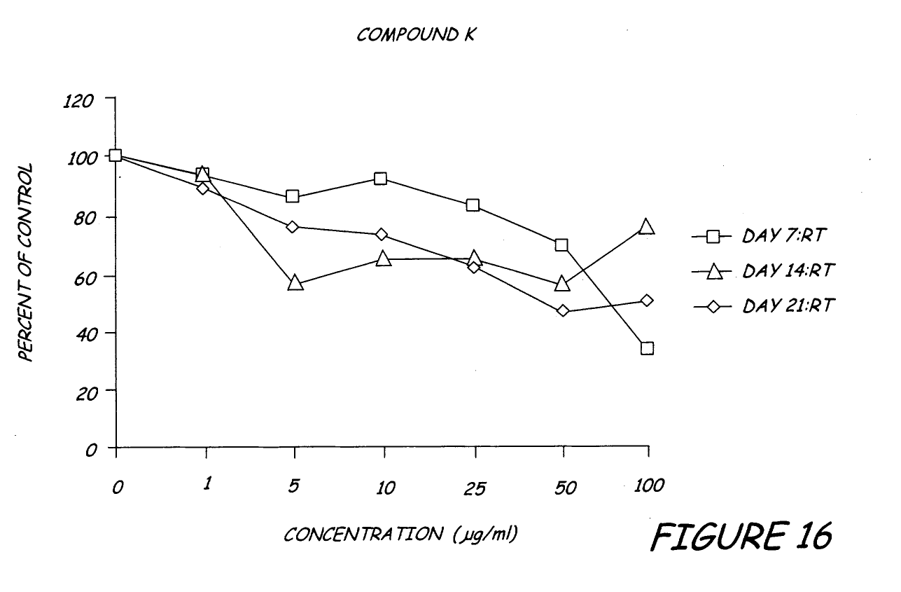

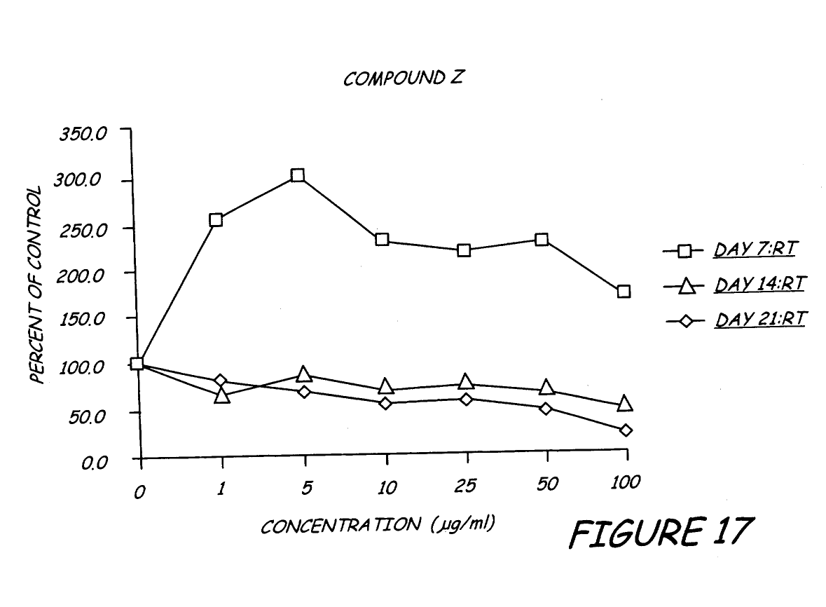

FIGS. 16 and 17 and Table 12 show the results of the exposure of chronically infected PBMCs to compound K and Z for 7, 14 and 21 days. Compound Z in chronically infected cells (FIG. 17) initially stimulated virus replication by 2 to 3-fold at day 7, while compound K (FIG. 16) inhibited replication with an IC₅₀ in chronically infected PBMCs of 68 μg/ml. Stimulation of virus replication by compound Z was reversed by day 14 of treatment and virus replication was suppressed with an IC₅₀ of approximately 30 μg/ml at 21 days, equivalent to its IC₅₀ in acutely infected PBMCs. Compound K reduced virus expression at day 7 (IC₅₀ 68 μg/ml) and 21 (IC₅₀ 32 to 100 μg/ml). However, for both compounds significant sustained suppression below 50% virus replication was not observed. Thus, the overall trend for both compounds appears to be increased virus inhibition with increased exposure to chronically infected PBMCs, however neither compound appears to be as potent inhibitor in this model as they are in acutely infected PBMCs, reducing virus replication significantly below that sustained by new rounds of infection.

4. Testing of the B and Z Compounds in n PBMC Time of Addition Assay

This time of addition assay measures reproducible events from initial virus binding to the trans activation of the β-galactosidase reporter by newly produced Tat protein. This is a single round of infection assay and is thus “cleaner” and more “precise” than a multi-round time of addition assay. Initial testing with the K and Z compound showed no significant cytotoxic effects of these compounds on the HeLa CD4 LTR β-gal cells, thus a time of addition assay was initiated. The K and Z compound failed to inhibit HIV replication in the assay. Since the time of addition assay is typically considered to be a high multiplicity of infection (M.O.I.) assay these results can be interpreted as either the antiviral target for the compounds was not present or the M.O.I. was too high and antiviral activity was not seen due to excess virus. Since it is hypothesized that the K and Z compounds work by interfering with virus transcription the later possibility seems more likely. Thus the use of a time of addition assay with PBMCs was utilized.

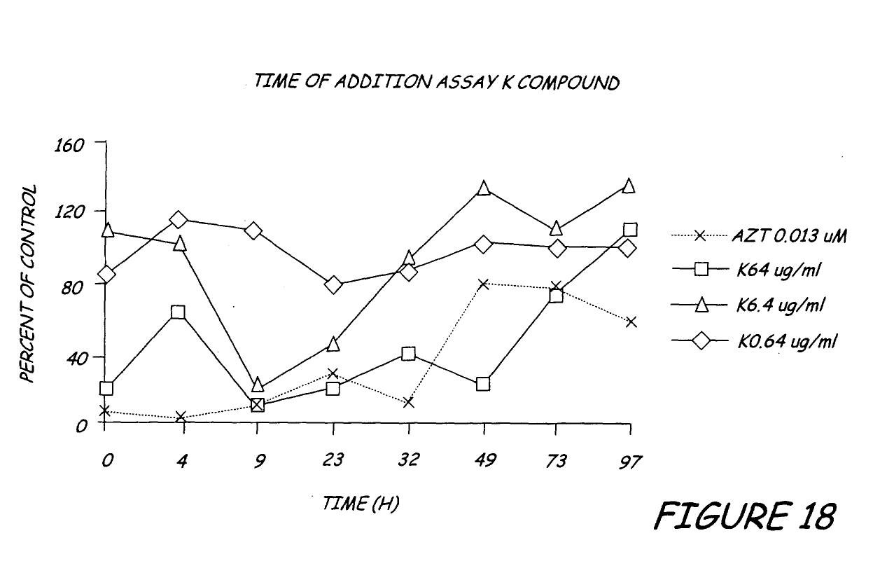

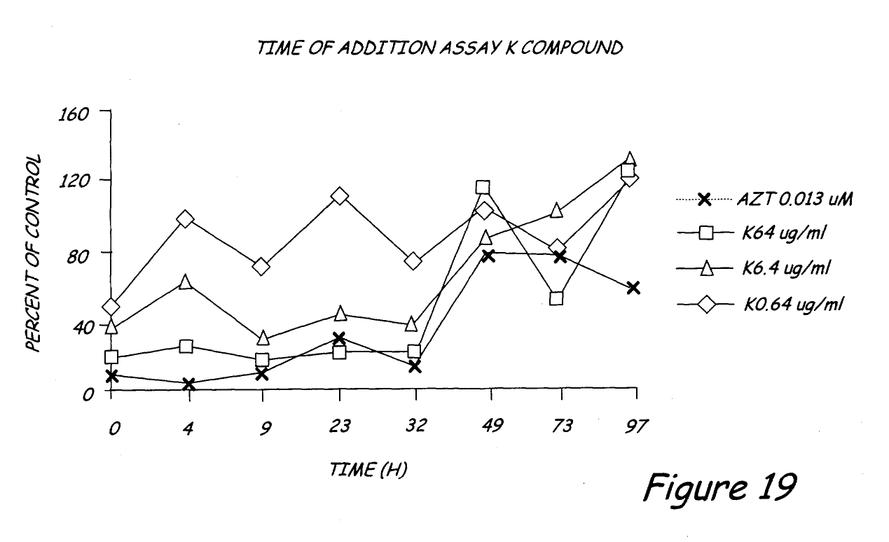

FIGS. 18 and 19 show the results of the time of addition assay with the K and Z compound. In time of addition assays the timed addition of compounds after initial infection allows the identification of specific stages of virus replication. This is seen with the positive control compound AZT. AZT is a reverse transcriptase inhibitor. As seen in the graphs addition of AZT after 32 h of infection has no significant effect on virus replication. Thus, at 32 h the infection has been established and further inhibition of RT activity does not alter over all virus replication. FIG. 18 shows the addition of compound K. Compound K maintained antiviral activity at 64 μg/ml (75% suppression of virus replication) when added as late as 49 h post infection. Thus it has an antiviral target that is occurring after completion of reverse transcription. Several features of the lower concentrations of compound K are important. In general since this time of addition uses multiple rounds of infection in the PBMCs versus a single round in cell lines systems, lower concentrations of compound will often shift the curve to the left. This is seen in FIG. 18. Another feature that is important is that inhibition of virus replication is enhanced by compound K at 9 h (87% suppression) versus 4 h (35% suppression) of addition at 64 μg/ml, and this is mimicked at 6.4 μg/ml (4 h no inhibition; 9 h 71% suppression). This suggests that antiviral activity for compound K may require the formation of a cellular/viral target before it is fully active, in contrast to this see the relative smooth curve for AZT. In toto, the data for compound K suggests an antiviral target after reverse transcription and in light of its activity in U1 and ACH-2 cell, this is potentially a post-integrative target.

The antiviral activity of compound Z in the time of addition assay, on the other hand, parallels that of AZT (FIG. 19). There is also a slight loss of activity at 4 h as seen with compound K, but it is not of the same magnitude. Because the PBMC time of addition assay is a multiple round infection assay, the separation between RT inhibitors and those with antiviral targets immediately after completion of RT is not as delineated as the single-round cell line-based models. Thus, from this data it was not determined if compound Z is preventing reverse transcription or works after completion of reverse transcription. From this data, it does not appear that the antiviral activity of compound Z is not associated with virus entry events.

Thus the time of addition assays suggest the compound K and Z are mediating their antiviral activities by different mechanisms of action and their antiviral targets arc associated with events during reverse transcription (Z) or following reverse transcription (K).

The data suggest that compound K has an antiviral target that is post integrative in nature. This evidenced by its ability to inhibit virus replication in the post-integrative U1 and ACM-2 cell line models. The time of addition assays support this observation by suggesting an antiviral target occurring after completion of reverse transcription. In contrast to these results compound K was only marginally active in chronically infected PBMCs. However previous work has shown that 50 to 60% of the total virus replication in these systems is due to new rounds of infection. Close examination of the data in FIG. 16 and Table 12 shows that treatment for 14 and 21 days results in suppression of virus replication in the 30 to 50% range from 5 to 100 μg/ml range. This suggests that the antiviral target for compound K may be more associated with acutely infected cells than chronically infected cells. However it does not rule out potential post-reverse transcription or post integration targets. The data for compound Z suggest that it is targeting an antiviral target different from compound K, and time of addition experiments and the results of the post-integrative U1/ACH-2 model suggest the target is either reverse transcription or a target closely related to reverse transcription.

Overall these results point to different mechanisms of action for compound K and Z. Additionally, both compound K and Z are more potent on acutely infected than chronically infected PBMCs, although they do display some potency against a chronically infected population. However these experiments cannot determine if this is due to inhibition of new rounds of infection or a decrease in the expression of virus from infected cells.

TABLE 12

| | Percent Control by Supernatant | | | | | |
|---|---|---|---|---|---|---|
| | Reverse Transcriptase activity | | | | | |
| Concentration | Day 7 | | Day 14 | | da 21 | |
| μg/ml) | K | Z | K | Z | K | Z |
| 0 | 100 | 100 | 100 | 100 | 100 | 100 |
| 1 | 94 | 256 | 94 | 63 | 90 | 80 |
| 5 | 87 | 303 | 57 | 87 | 77 | 70 |
| 10 | 92 | 230 | 66 | 69 | 74 | 56 |
| 25 | 84 | 217 | 65 | 74 | 63 | 60 |
| 50 | 70 | 229 | 56 | 67 | 46 | 48 |
| 100 | 34 | 167 | 75 | 49 | 50 | 22 |

Chronically Infected CEMISK-1 Cell Assay:

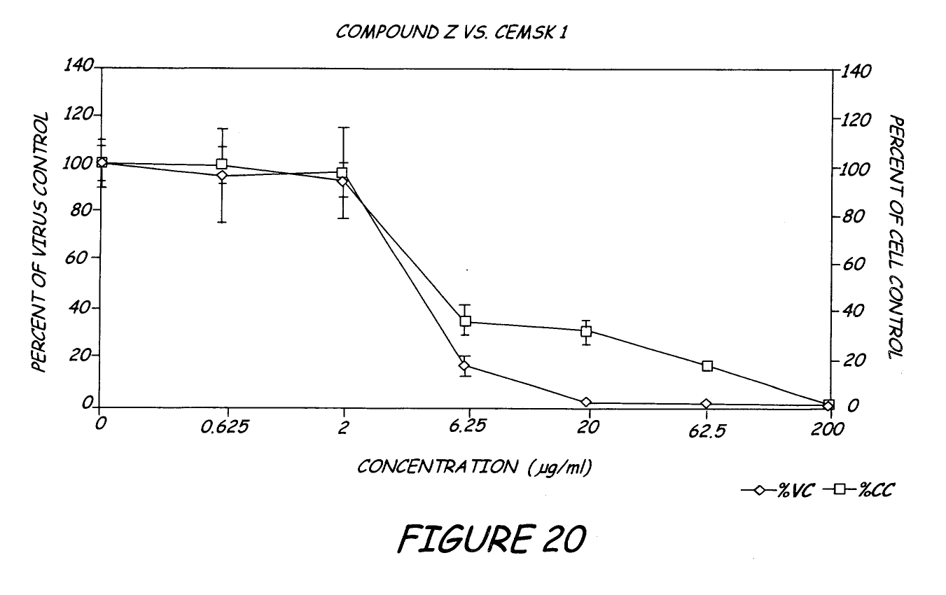

Fifty thousand (5.0×104) CEM-SS cells chronically infected with the SK-1 strain of HIV in exponential growth were placed into 0.2 cm wells in a total volume of 200 ul with or without serial dilutions of compound. The chronically infected CEM cells were cultured and the assay performed in RPMI 1640 supplemental with 10% Fetal Bovine Serum (heat inactivated), 2 mM L-glutamine, 100 U/mL penicillin, 100 U/mL streptomycin and 10 ug/mL gentamycin. On day 3 cell-free supernatants were collected and cell viability measured by MTS dye reduction. (See Table 13 and FIG. 20).

TABLE 13
Compound Z Versus CEMSKI Chronic Infection

| | | | | | | | |
|---|---|---|---|---|---|---|---|
| CONC (μG/Ml) | 0 | 0.625 | 2 | 6.25 | 20 | 62.5 | 200 |

RT VALUES (cpm)

| | | | | | | | |
|---|---|---|---|---|---|---|---|
| SAMPLE t | 11090.7 | 9567.7 | 8509.7 | 2189.7 | 130.7 | 62.7 | 2.7 |
| SAMPLE 2 | 9683.7 | 8192.7 | 9639.7 | 1692.7 | 130.7 | 66.7 | 74.7 |
| SAMPLE 3 | 10762.7 | 12230.7 | 12386.7 | 1400.7 | 122.7 | 182.7 | 66.7 |
| MEAN | 10512.3 | 9997.0 | 10178.7 | 1761.0 | 128.0 | 104.0 | 48.0 |
| % VC | 100.0 | 95.1 | 96.8 | 16.8 | 1.2 | 1.0 | 0.5 |
| STD DEV | 7.0 | 19.5 | 19.0 | 3.8 | 0.0 | 0.6 | 0.4 |

TOXICITY VALUES (MTS - O. D. @ 490/650 nm)

| | | | | | | | |
|---|---|---|---|---|---|---|---|
| SAMPLE 1 | 1.568 | 1.606 | 1.525 | 0.532 | 0.441 | 0.342 | 0.003 |
| SAMPLE 2 | 1.903 | 1.835 | 1.679 | 0.749 | 0.624 | 0.274 | 0.009 |
| SAMPLE 3 | 1.807 | 1.829 | 1.760 | 0.611 | 0.594 | 0.287 | 0.022 |
| MEAN | 1.759 | 1.757 | 1.655 | 0.631 | 0.553 | 0.301 | 0.011 |
| % CC | 100.0 | 99.8 | 94.1 | 35.8 | 31.4 | 17.1 | 0.6 |
| STD-DEY | 9.8 | 7.4 | 6.8 | 6.2 | 5.6 | 2.1 | 0.6 |

IC₅₀ (μG/Ml) = 4.49
ITC₅₀ (ug/ml) = 5.22
Tl = 1.16

Inhibition of Attachment Assay

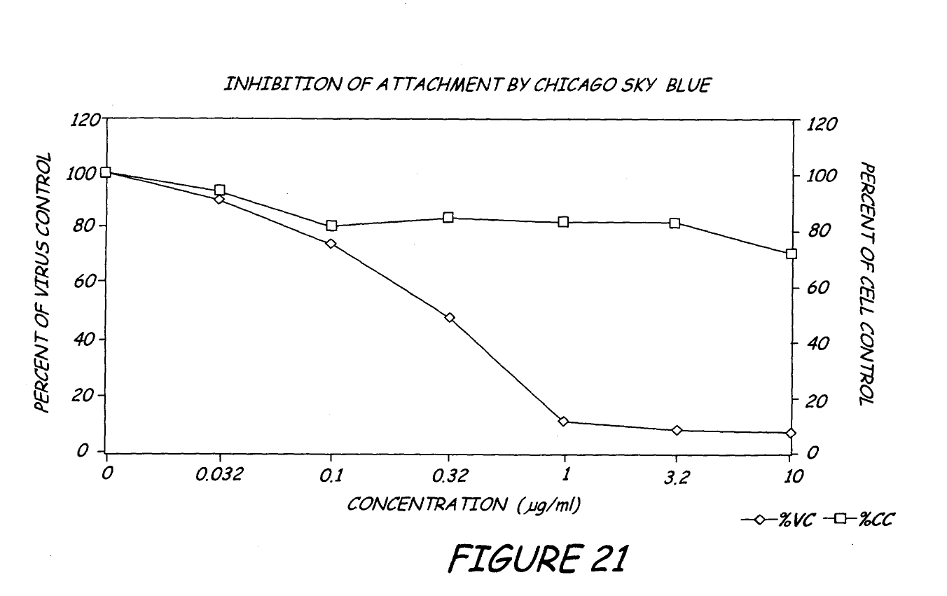

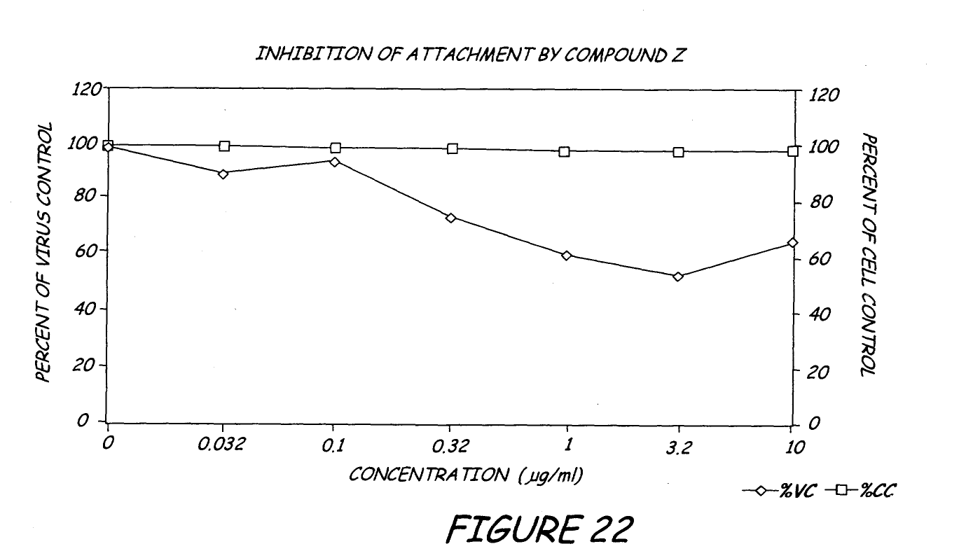

The attachment assay was performed with the HeLa CD4 LTR B-gal cells available from the AIDS Research and Reference Reagent Repository. HeLa cells do not express cell surface CD4, express the HIV coreceptor CXCR4 and are not infectable by HIV-1 unless CD4 is present. HeLa CD4 LTR B-gal cells express cell surface CD4 and contain an LTR B-galactosidase reporter construct. Upon infection either the Tat protein incorporated into the virion or new Tat produced following virus integration and transcription trans-activate the LTR B-gal reporter, leading to expression of the B-galactosidase enzyme. HeLa CD4 LTR B-gal cells are routinely cultured with the required selection antibiotics and scrended for mycoplasma contamination. Twenty-four h prior to initiation of the assay cells were trypsinized, counted and 1×104 cells placed in a 0.2 cm well in media without selection antibiotics. Media was removed and compound media placed on the cells and incubated for 15 to 30 min at 37° C. A known titer of virus was then added to the wells and the incubation continued for 1 h. At the end of the incubation the wells were washed 6 times with media and the culture continued for 48 h. At 48 h the wells were washed one time with PBS and B-galactosidase enzyme expression determined by chemiluminescence per manufacturers instructions (Tropix Gal-screen TM, Tropix, Bedford, Mass.). Compound toxicity was monitored on sister plates using MTS dye reduction. (See Tables 14 through 16 and FIGS. 21 and 22).

TABLE 14

| COMPOUND | IC₅₀ | TC₅₀ | Therapeutic Index |
|---|---|---|---|
| Chicago Sky Blue | 0.30 μg/mL | >10 μg/mL | >33 |
| Compound Z | >100 μg/mL | Not done | N/A |

TABLE 15
INHIBITION OF ATTACHMENT BY CHICAGO SKY BLUE

| | | | | | | | |
|---|---|---|---|---|---|---|---|
| CONC (dilution) | 0 | 0.032 | 0.1 | 0.32 | 1 | 3.2 | 10 |

RLU (Relative Light Units)

| | | | | | | | |
|---|---|---|---|---|---|---|---|
| SAMPLE 1 | 160442.3 | 133472.3 | 87897.3 | 44529.3 | 11599.3 | 5830.3 | 13006.3 |
| SAMPLE 2 | 145604.3 | 151379.3 | 120976.3 | 74986.3 | 11726.3 | 16157.3 | 6367.3 |
| SAMPLE 3 | 117076.3 | 98412.3 | 106442.3 | 84351.3 | 22764.3 | 13272.3 | 14426.3 |
| MEAN | 141041.0 | 127754.7 | 105105.3 | 67955.7 | 15363.3 | 11753.3 | 11266.7 |
| % VC | 100.0 | 90.6 | 74.5 | 48.2 | 10.9 | 8.3 | 8.0 |

TOXICITY VALUES (Cell Titer - O. D. @ 4901650 nm)

| | | | | | | | |
|---|---|---|---|---|---|---|---|
| SAMPLE 1 | 0.989 | 0.989 | 0.989 | 0.989 | 0.989 | 0.989 | 0.989 |
| SAMPLE 2 | 1.432 | 1.332 | 0.989 | 0.989 | 0.989 | 0.989 | 0.989 |
| SAMPLE 3 | 1.627 | 1.457 | 1.293 | 1.449 | 1.366 | 1.392 | 0.989 |
| MEAN | 1.349 | 1.259 | 1.090 | 1.142 | 1.115 | 1.123 | 0.989 |
| % CC | 100.0 | 93.3 | 80.8 | 84.7 | 82.6 | 83.3 | 73.3 |

IC₅₀ (μG/mL) = 0.30
TC₅₀ (μG/mL) = >.10
TI = >33

TABLE 16
INHIBITION OF ATTACHMENT COMPOUND Z
RLU (Relative Light Units)

| CONC (dilution) | 0 | 0.32 | 1 | 3.2 | 10 | 32 | 100 |
|---|---|---|---|---|---|---|---|
| SAMPLE 1 | 157093.7 | 130319.7 | 149854.7 | 127658.7 | 108096.7 | 83041.7 | 108723.7 |
| SAMPLE 2. | 151736.7 | 146305.7 | 145831.7 | 103018.7 | 93093.7 | 99936.7 | 96222.7 |
| SAMPLE 3 | 147890.7 | 135339.7 | 137409.7 | 109616.7 | 83971.7 | 65609.7 | 100048.7 |
| MEAN | 152240.3 | 137321.7 | 144365.3 | 113431.3 | 95054.0 | 82862.7 | 101665.0 |
| % VC | 100.0 | 90.2 | 94.8 | 74.5 | 62.4 | 54.4 | 66.8 |

TOXICITY VALUES (Colt Titer - O. D. @ 490/650 nm)

| CONC (μG/Ml) | 0 | 0.32 | 1 | 3.2 | 10 | 32 | 100 |
|---|---|---|---|---|---|---|---|
| SAMPLE 1 | 0.989 | 0.989 | 0.989 | 0.969 | 0.989 | 0.989 | 0.989 |
| SAMPLE 2 | 0.989 | 0.989 | 0.989 | 0.989 | 0.989 | 0.989 | 0.989 |
| SAMPLE 3 | 0.989 | 0.989 | 0.989 | 0.989 | 0.989 | 0.989 | 0.989 |
| MEAN | 0.989 | 0.989 | 0.989 | 0.989 | 0.989 | 0.989 | 0.989 |
| % CC | 100.0 | 100.0 | 100.0 | 100.0 | 100.0 | 100.0 | 100.0 |

IC₅₀ (μG/mL) = >100
TC₅₀ (μG/mL) + >100
TI = N/A

One of ordinary skill in the art will appreciate further features and advantages of the invention based on the above-described embodiments. Accordingly, the invention is not to be limited by what has been particularly shown and described, except as indicated by the appended claims. All publications and references cited herein, including those in the background, are expressly incorporated herein by reference in their entirety.

I claim:

1. A method for treating human immunodeficiency virus (HIV) comprising the step of administering a therapeutically effective amount of an iodiscorbate and pharmaceutically acceptable salts thereof to a subject in need thereof.

2. The method of claim 1, wherein the iodiscorbate has a formula of XISrC₆H₅O₆, wherein X is selected from the group consisting of bismuth, potassium, and zinc.

3. The method of claim 2, wherein the iodiscorbate has the formula BiISrC₆H₅O₆.

4. The method of claim 2, wherein the iodiscorbate has the formula K₂ISrC₆H₅O₆.

5. The method of claim 2, wherein the iodiscorbate has the formula ZnISrC₆H₅O₆.

6. The method of claim 1, wherein the iodiscorbate is a compound including operably bonded iodine, ascorbic acid, strontium, and one member selected from the group consisting of bismuth, potassium, and zinc.

7. The method of claim 1, wherein the treatment inhibits viral reverse transcription.

8. The method of claim 1, wherein the treatment prevents viral reverse transcription.

9. The method of claim 1, wherein the treatment inhibits viral post-reverse transcription.

10. The method of claim 1, wherein the treatment prevents viral post-reverse transcription.

11. The method of claim 1, wherein the treatment inhibits viral integration.

12. The method of claim 1, wherein the treatment prevents viral integration.

13. The method of claim 1, wherein the treatment inhibits viral post-integration.

14. The method of claim 1, wherein the treatment prevents viral post-integration.

15. The method of claim 1, wherein the subject is a human.

16. The method of claim 1, further comprising the step of administering a protease inhibitor in combination with the iodiscorbate.

17. The method of claim 16, wherein the protease inhibitor is selected from the group consisting of saquinavir, ritonavir, indinavir, nelfinavir, and amprenavir.

18. The method of claim 1, further comprising the step of administering a nucleoside analog reverse transcriptase inhibitor in combination with the iodiscorbate.

19. The method of claim 18, wherein the nucleoside analog reverse transcriptase inhibitor is selected from the group consisting of zidovudine, didanosine, zalcitabine, stavudine, lamivudine, and abacavir.

20. The method of claim 1, further comprising the step of administering a non-nucleoside reverse transcriptase inhibitor in combination with the iodiscorbate.

21. The method of claim 20, wherein the non-nucleoside reverse transcriptase inhibitor is selected from the group consisting of nevirapine, efavirenz, and delavirdine.

22. A packaged pharmaceutical composition for treating human immunodeficiency virus (HIV) in a subject, comprising
a container holding a therapeutically effective amount of an iodiscorbate and pharmaceutically acceptable salts thereof; and
instructions for using the iodiscorbate for treating HIV in the subject.

23. A packaged pharmaceutical composition for treating human immunodeficiency virus (HIV) in a subject, comprising
a container holding a therapeutically effective combination of an iodiscorbate and pharmaceutically acceptable salts thereof and a nucleoside analog reverse transcriptase; and
instructions for using the combination of the iodiscorbate and nucleoside analog reverse transcriptase for treating HIV in the subject.

24. A packaged pharmaceutical composition for treating human immunodeficiency virus (HIV) in a subject, comprising
a container holding a therapeutically effective combination of an iodiscorbate and pharmaceutically acceptable salts thereof and a non-nucleoside reverse transcriptase inhibitor; and
instructions for using the combination of the iodiscorbate and non-nucleoside reverse transcriptase inhibitor for treating HIV in the subject.

25. A pharmaceutical composition comprising an iodiscorbate and a pharmaceutical acceptable carrier.
